(19) United States
(12) Patent Application Publication
(10) Pub. No.: US 2010/0286514 A1
(43) Pub. Date: Nov. 11, 2010

(54) CONTRAST ENHANCEMENT BETWEEN LINEAR AND NONLINEAR SCATTERERS

(75) Inventors: Timothy Grant Leighton, West Wellow (GB); Paul Robert White, Southampton (GB); Daniel Clark Finfer, Southam (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF SOUTHAMPTON, Southampton (GB)

(21) Appl. No.: 11/917,990

(22) PCT Filed: Jun. 26, 2004
(86) PCT No.: PCT/GB06/02335

§ 371 Date: Jul. 16, 2010

(30) Foreign Application Priority Data
Jun. 25, 2005 (GB) 0513031.5

(51) Int. Cl. A61B 8/00

(52) U.S. Cl. 600/437

(57) ABSTRACT

A method for creating an acoustic observation of a target volume, the method comprising the steps of transmitting a group of at least two acoustic pulses towards the target volume, receiving at least one detector an echo of the group scattered from the target volume, the echo having linear and nonlinear components, processing the scattered signal in such a way as to enhance at least part of the nonlinear component (and suppress the linear component) of the scattered signal in a signal P₊, processing the scattered signal in such a way as to suppress at least part of the nonlinear component (and enhance the linear component) of the scattered signal in a signal P₋, and producing a detection signal from a mathematical combination of the signals P₊ and P₋. The method can be used to suppress at least part of the non-linear component, and to enhance the linear component in order to improve the contrast of an image of an object located in water and surrounded by an oceanic bubble cloud, by utilising a ratio of signals P+ and P−.

In other situations where bubbles are required to be enhanced in the image, such as in biomedical ultrasonics, the nonlinear component can be arranged to be enhanced, and the linear component suppressed, by utilising the ratio P−/P+.

The present invention relates to contrast enhancement between linear and nonlinear scatterers in a transmitting/receiving apparatus that observes a target by transmitting a pulsed signal towards the target and monitors the receipt of signals scattered by the target.

Throughout this specification, a ‘pulse’ or ‘pulsed signal’ is defined as any waveform of finite duration (including near-tonal pulses shaped by some envelope function, or chirps, or pseudorandom noise sequences, or M-sequences). The characteristics of the ‘pulse’ (such as its centre frequency or bandwidth) may of course change between one group (e.g. pair, trio etc) of TWIPS pulses and the following group.

The apparatus may be monostatic (source and receiver located at the same place), or bistatic or multistatic (source and receiver(s) situated at different locations).

The present invention in some preferred embodiments relates to acoustic detection, and in particular to observations in environments containing bubbles.

The invention relates most particularly, but not exclusively, to liquid environments containing gas bubbles, and for those environments to observations using acoustic and ultrasonic techniques.

Some aspects of the invention relate to the use of electromagnetic radiation, such as in RADAR and LIDAR applications of the invention.

The term ‘bubble’ will be used herein to include actual bubbles, but where appropriate to include other systems that scatter waves nonlinearly, such as an underground or in-tissue gas body or, for electromagnetic waves, certain types of circuit or junction.

According to a first aspect of the invention we provide a method for creating an acoustic observation of a target volume, the target volume comprising at least one bubble, the method comprising transmitting a group of at least two acoustic pulses towards the target volume, the group of pulses being arranged such that the bubble will scatter the group in a nonlinear manner, receiving at least one detector an echo of the group of pulses scattered from the target volume, and processing the received scattered pulses in such a way as to modify at least part of the nonlinear component of the scattered pulses in the detection signal, wherein the time between the centre of a first pulse of the group and the centre of a second pulse of the group is longer than half of the characteristic decay time of the signal between the pulses.

This method has particular application to the use of sonar in oceanic bubble clouds.

The oscillatory frequency within each pulse is chosen to be appropriate for inducing sufficient nonlinearity in enough of the bubbles present in the target volume commensurate with the level of detection enhancement required.

Whilst for the special case of a monodisperse or near-monodisperse bubble population (as is found with some contrast agents) the degree of nonlinearity in the bubble response and the performance of TWIPS are enhanced when the insonification frequency is close to the bubble resonance, in general in oceanic and industrial environments (and for the two oceanic sonar examples simulated, and experimentally tested, in the technical description provided later in this patent), there will be a wide distribution of bubble sizes present, and in such circumstances the performance of TWIPS (Twin Inverted Pulse Sonar) is enhanced if the oscillatory frequency is lower than the resonance frequencies of the majority of bubbles which contribute to the scatter and re-radiation (see the technical report under “The radiated pressures”).

The effect is continuous, such that reducing the frequency and increasing the drive amplitude will tend to increase the nonlinearity in the scatter. However there will be balancing considerations, such as the reduction in spatial resolution associated with lower frequencies, and the generation of cavitation at the transducer associated with increasing drive amplitudes (an effect which is suppressed by increasing water depths, although this in turn can reduce the degree of nonlinearity excited in the bubbles). For most oceanic bubble populations within a few metres of the ocean surface, in order to excite sufficient nonlinearity in the oceanic bubble population, the zero-to-peak acoustic pressure amplitude of the incident pulses is preferably greater than about 10 kPa, and the drive frequency is preferably (but not necessarily—see section “Concluding remarks” of the Technical report) below about 100 kHz (depending on the bubble size distribution). For the examples in the simulation and experiment included in the Technical report, a frequency of below 20 kHz was used. Other environments or applications (such as biomedical contrast agents) will require commensurately adjusted frequencies and amplitudes. The performance is a continuum, with TWIPS still potentially operating at higher frequencies and lower pressures, the reduction in nonlinearity being offset by an improvement in, for example, resolution. In applications where the bubble size distribution is not so broad (e.g. biomedical ultrasonics), much greater frequencies (<100 MHz) can be used. In electromagnetic applications (RADAR, LIDAR etc.) commensurately higher frequencies can be used.

Performance will tend to improve as the amplitude increases, but as most practical sonar avoids the generation of inertial cavitation, within about 5 m of the ocean surface this will place an upper limit on the amplitude of about 150 kPa. Hence, at this example location, the group of pulses preferably has a peak amplitude between 10 kPa and 150 kPa. The allowable upper limit will increase as the transducers are placed at greater water depths.

The target volume may comprise more than one bubble in the form of a bubble cloud, plus a linearly scattering target, the group of pulses being arranged such that a sufficient number of the bubbles in the cloud respond to the group of pulses in a nonlinear manner to achieve the desired level of performance enhancement.

The second pulse of the group of acoustic pulses is preferably substantially identical to the first pulse but of opposite polarity. The acoustic signal is most preferably of the form P(t)=Γ(t)−Γ(t−t₁), where Γ is a pressure function, t is time, and t₁ corresponds to the time delay between the two pulses.

Preferably said part of the nonlinear component is suppressed in the detection signal. An object within or behind the bubble cloud will scatter acoustic energy in a substantially linear way, and so suppression of part of the nonlinear component provides a clearer observation of the object hidden within the bubble cloud. For example, in underwater sonar, a mine within a bubble cloud could be detected using this method.

Said part of the nonlinear component is preferably suppressed by filtering the received signal substantially according to the function h(t)=δ(t)−δ(t+t₁) where δ represents the Dirac delta function, and t is time. This is equivalent to shifting the received signal by a time t₁ and then subtracting the shifted signal from the received signal in order to substantially eliminate the even powered nonlinear components of the signal from the time window of interest (ie the overlap region). (Throughout this report, illustrative references are made to a power series expansion of the nonlinearity in the bubble response and radiation. It is recognised that this is just one form of representation, and references to the even- and odd-powered terms will be taken to apply to the asymmetric and symmetric terms in a general expression of the nonlinearity). The net result corresponds to splitting the received time series in half and subtracting one half from the other. The resulting signal is referred to as P₋(t).

Alternatively, part of the nonlinear component may be enhanced in the detection signal. This will provide a clearer observation of the bubbles for greater contrast with the remainder of the target volume. For example, in biomedical contrast agent imaging, bubbles may be injected into the blood stream in order to provide greater contrast between the blood and the surrounding tissue.

The part of the nonlinear component is preferably enhanced by filtering the received signal substantially according to the function h(t)=δ(t)+δ(t+t₁).

This is equivalent to shifting the received signal by a time t₁ and then adding the shifted signal to the received signal in order to substantially eliminate the linear and odd powered nonlinear components of the signal from the time window of interest (ie the overlap region). The net result corresponds to splitting the received time series in half and adding the two halves together. The resulting signal is referred to as P₊(t).

According to a second aspect of the invention we provide a method for creating an acoustic observation of a target volume, the method comprising transmitting a group of at least two acoustic pulses towards the target volume, receiving at least one detector an echo of the group of pulses scattered from the target volume, the echo having linear and nonlinear components, and processing the received scattered pulses in such a way as to suppress at least part of the nonlinear component of the scattered pulses in the detection signal, wherein the time between the centre of a first pulse of the group and the centre of a second pulse of the group is greater than half of the characteristic decay time of the signal between the pulses.

For use in the specific application of sonar in bubbly ocean clouds, this would correspond to intervals of greater than 10 μs. Commensurately smaller minimum intervals would be required for other applications, eg biomedical ultrasonics, RADAR, LIDAR etc.

Preferably the linear components and the remainder of the nonlinear components of the scattered pulses are also enhanced.

The second pulse of the group of acoustic pulses is preferably substantially identical to the first pulse but of opposite polarity. The acoustic signal is most preferably of the form P(t)=Γ(t)−Γ(t−t₁), where Γ is a pressure function, t is time, and t₁ corresponds to the time delay between the two pulses.

Said part of the nonlinear component is preferably suppressed by filtering the received signal substantially according to the function h(t)=δ(t)−δ(t+t₁) where δ represents the Dirac delta function, and t is time.

Preferably the target volume comprises at least one object, or a plurality of objects such as a bubble cloud, which together are responsible for the majority of the nonlinear component of the scattered signal.

According to a third aspect of the invention we provide a method for creating an acoustic observation of a target volume, the method comprising the steps of transmitting a group of at least two acoustic pulses towards the target volume, receiving at least one detector an echo of the group scattered from the target volume, the echo having linear and nonlinear components, processing the scattered signal in such a way as to enhance at least part of the nonlinear component (and preferably suppress the linear component, and the remainder of the nonlinear component) of the scattered signal in a signal P₊, processing the scattered signal in such a way as to suppress at least part of the nonlinear component (and preferably enhance the linear component, and the remainder of the nonlinear component) of the scattered signal in a signal P₋, and producing the detection signal from a mathematical combination of the signals P₊ and P₋

The second pulse of the group of acoustic pulses is preferably substantially identical to the first pulse but of opposite polarity. The acoustic signal is most preferably of the form P(t)=Γ(t)−Γ(t−t₁), where Γ is a pressure function, t is time, and t₁ corresponds to the time delay between the two pulses.

Preferably the mathematical combination is a ratio.

In one embodiment the ratio P₊/P₋ is taken in order to further enhance at least part of the nonlinear component, and suppress the linear component, and the remainder of the nonlinear component of the scattered signal, in the observation.

In another embodiment the ratio P₋/P₊ may be taken in order to further suppress at least part of the nonlinear component, and enhance the linear component, and the remainder of the nonlinear component of the scattered signal, in the observation.

It is recognised that use of the ratio P₊/P₋, whilst potentially greatly increasing the contrast of some echoes, introduces greater instability than if P₊ is used alone. Therefore this embodiment preferably includes other signals formed by combining mathematical combinations of P₊ and P₋, for example by multiplying the ratio P₊/P₋ by P₊ (or, for example, the squares of these) to combine elements of both the enhanced detection of P₊/P₋ with the stability of P₊ in enhancing at least part of the nonlinear component (and suppress the linear component, and the remainder of the nonlinear component) of the reflected scattered signal in the observation. Another example of such a function could involve summations, for example involving a weighted summation of P₊/P₋ and P₊, or powers thereof. Specific examples of this include stabilisation through the addition of a function or constant to the denominator of the ratio (through, for example, the formation of P₊/(P₋+P₊) to enhance bubbles, or P₋/(P₋+P₊) to enhance linear targets).

It is recognised that use of the ratio P₋/P₊, whilst potentially greatly increasing the contrast of some echoes, introduces greater instability than if P₊ or P₋ are used alone. Therefore this embodiment preferably includes other signals formed by combining mathematical combinations of P₊ and P₋, for example by multiplying the ratio P₋/P₊ by P₋ (or, for example, the squares of these) to combine elements of both the enhanced detection of P₋/P₊ with the stability of P₋ in suppressing at least part of the nonlinear component (and enhancing the linear component, and the remainder of the nonlinear component) of the reflected scattered signal in the observation. Another example of such a function could involve summations, for example involving a weighted summation of P₋/P₊ and P₋, or powers thereof.

Preferably the target volume comprises at least one object, or a plurality of objects such as a bubble cloud, which together are responsible for the majority of the nonlinear component of the scattered signal. The degree to which a bubble scatters nonlinearly depends on several parameters, primarily the amplitude and frequency of the driving field, and the bubble size. The wider the range of bubble sizes present, the more difficult it is in general to excite nonlinearities from the whole bubble population. Whilst increasing the amplitude of the driving pulse tends to increase the nonlinearity, there are practical limitations to this resulting from transducer technology and cavitation inception. The frequency must therefore be appropriate to the bubble population. When the population contains a wide distribution of sizes, such as in the ocean, for practical pulse amplitudes we prefer to use a frequency of less than about 100 kHz.

Having excited a sufficient degree of nonlinearity, the detection enhancement scheme exploits this through the use of pairs of consecutive pulses, whereby within each pair one pulse is delayed with respect to the other by more than half of the characteristic decay time of the signal between the pulses. For use in the specific application of sonar in bubbly ocean clouds, this would correspond to intervals of greater than 10 μs. Commensurately smaller minimum intervals would be required for other applications, eg biomedical ultrasonics, RADAR, LIDAR etc.

According to a fourth aspect of the invention we provide apparatus for creating an acoustic observation of a target volume in accordance with the method of any one of the preceding claims, the apparatus comprising at least one acoustic pulse transmitter and at least one acoustic pulse receiver, a signal processing unit responsive to the output of the receiver, the signal processing unit being so configured as in use to enhance at least part of the nonlinear component (and suppress the linear component) of the scattered signal to produce a signal P₊, and also to suppress at least part of the nonlinear component (and enhance the linear component) of the scattered signal to produce a signal P₋, and a combiner unit arranged to produce in use a detection signal by mathematically combining the signals P₊ and P₋ in a manner such as to further enhance the contrast between said part of the nonlinear component and the linear component.

According to a fifth aspect of the invention we provide apparatus for creating an acoustic observation of a target volume in a human or animal body, the apparatus comprising an acoustic pulse transmitter and an acoustic pulse receiver adapted to be positioned adjacent to a human or animal body, a signal processing unit responsive to the output of the receiver, the signal processing unit being so configured as in use to enhance at least part of the nonlinear component (and suppress the linear component) of the scattered signal from the target volume to produce a signal P₊, and also to suppress at least part of the nonlinear component (and enhance the linear component) of the scattered signal from the target volume to produce a signal P₋, and a combiner unit arranged to produce in use a detection signal by mathematically combining the signals P₊ and P₋ in a manner such as to further enhance the contrast between said part of the nonlinear component and the linear component.

According to a sixth aspect of the invention we provide a transmitting/receiving apparatus for observing a target by transmitting a pulsed electromagnetic signal towards the target and monitoring the receipt of signals scattered by the target, the transmitter being arranged to transmit a group of at least two pulses towards the target volume, the group of pulses being so configured that the scattered signal comprises linear and nonlinear components, the detector being arranged to process the scattered pulses resulting from said group in such a way as to modify the appearance of at least part of the nonlinear component of the scattered pulses in the receiver output signal.

The electromagnetic signals may be RADAR signals, or LIDAR signals, for example.

In one embodiment said part of the nonlinear component of the scattered electromagnetic pulses is suppressed in the receiver output signal.

Alternatively, in another embodiment said part of the nonlinear component of the scattered electromagnetic pulses is enhanced.

In yet another, preferred, embodiment a first receiver signal P₊ is produced by the receiver by processing the received scattered signal so as to enhance part of the nonlinear component of the scattered electromagnetic pulses (and preferably suppress the linear component, and the remainder of the nonlinear component) and a second receiver signal P₋ is produced by processing the received scattered signal in such a way as to suppress at least part of the nonlinear component (and preferably enhance the linear component, and the remainder of the nonlinear component), and a receiver output signal is produced from a mathematical combination of the signals P₊ and P₋.

Embodiments of the invention will now be described, by way of example only, with reference to the field of underwater sonar, although it should also be understood that this field is by way of example only. Reference will be made to the accompanying Figures:

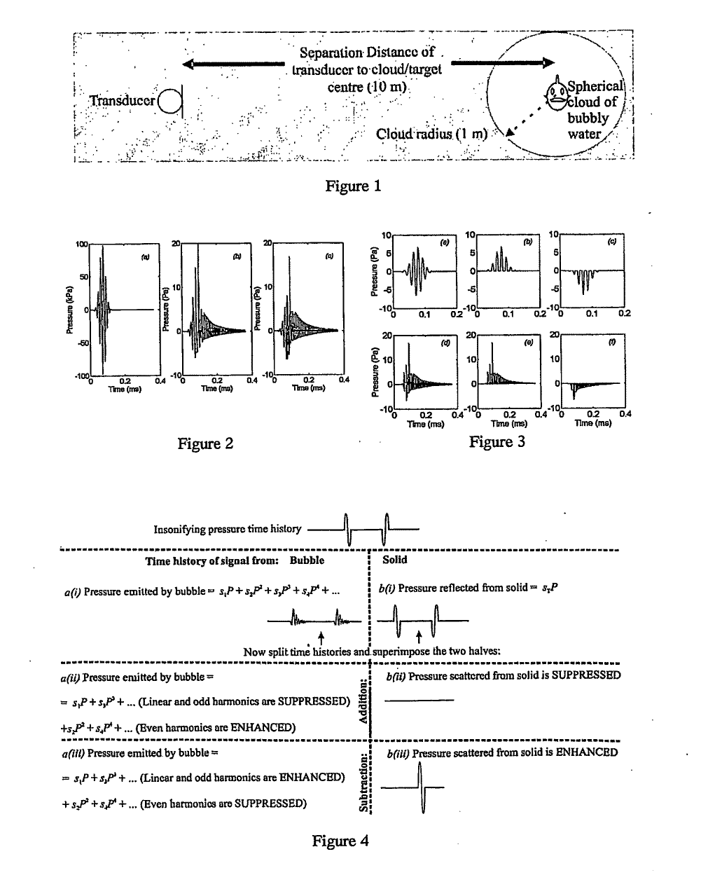

FIG. 1 is a schematic showing a typical problem, where a sonar source (the transducer on the left) is situated some distance (here 10 m) from a bubble cloud (which here has radius 1 m), and is trying to detect a target (here a fish) which is hidden in the bubble cloud. This is the geometry used in the simulations described hereafter.

FIG. 2 (a) The driving pulse (centre frequency 65.7 kHz) used to simulate the scatter shown in FIGS. 2(b), 2(c) and 3. Part (b) shows a simulation of the pressure detected at 1 m from target used in FIG. 3(a)-(c), and the bubble used in FIG. 3(d)-(f). Part (c) shows a superimposition of plot of FIG. 3(a) (thick black line) on plot of FIG. 2(b) (thin line).

FIG. 3 Calculations of pressures radiated by bubbles. (a) Pressure 1 m from linearly scattering target insonified by pulse of FIG. 2(a). Positive (b) and negative (c) half-wave rectification of (a) are shown. (d) Pressure at 1 m from air bubble (22.5 μm radius water under 1 bar static pressure) insonified by pulse of FIG. 2(a). Positive (e) and negative (f) half-wave rectification of (d) are shown.

FIG. 4 is a schematic for the operation of Twin Inverted Pulse Sonar (TWIPS).

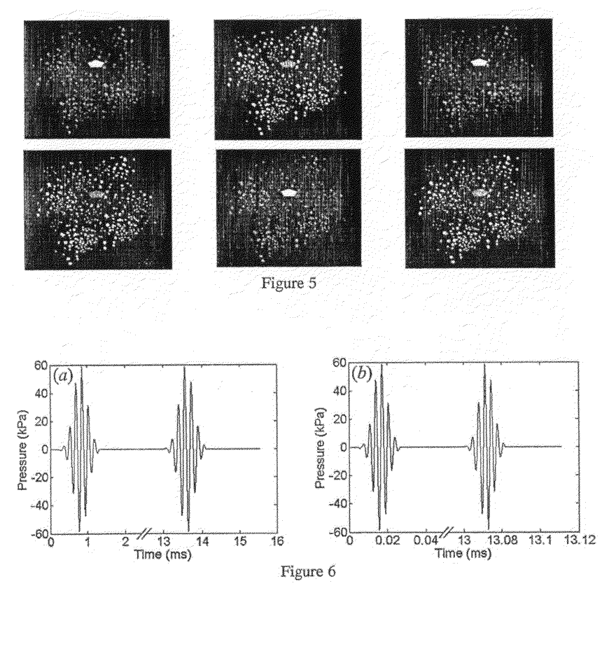

FIG. 5 by switching P₋ and P₊, or between P₋/P₊ and P₊/P₋ (or other mathematical combinations such as P₊²/P₋² and P₋²/P₊²), successive frames switch between enhancing the nonlinear scattering and the linear scattering. In this way, bubbles and linear targets (for example) appear to flash, increasing their visibility. The output is here shown in the form of an image, although this ‘switching’ facility is not restricted to images only, and could readily be implemented via simple time histories.

FIG. 6 The wavetrains used to insonify the marine environment in the particular implementation of TWIPS used in the simulation. There are of course an infinite number of wave types which can form a pair identical except for having opposite polarity. Hence this invention is not restricted to the specifics shown in this figure (eg number of cycles and envelope). Simulations were carried out for pulses based on centre frequencies of (a) 6 kHz and (b) 300 kHz. In changing the driving frequency from 6 kHz to 300 kHz in the simulation, the carrier and envelope signals increase in frequency, but the number of cycles within each pulse pair remains unchanged.

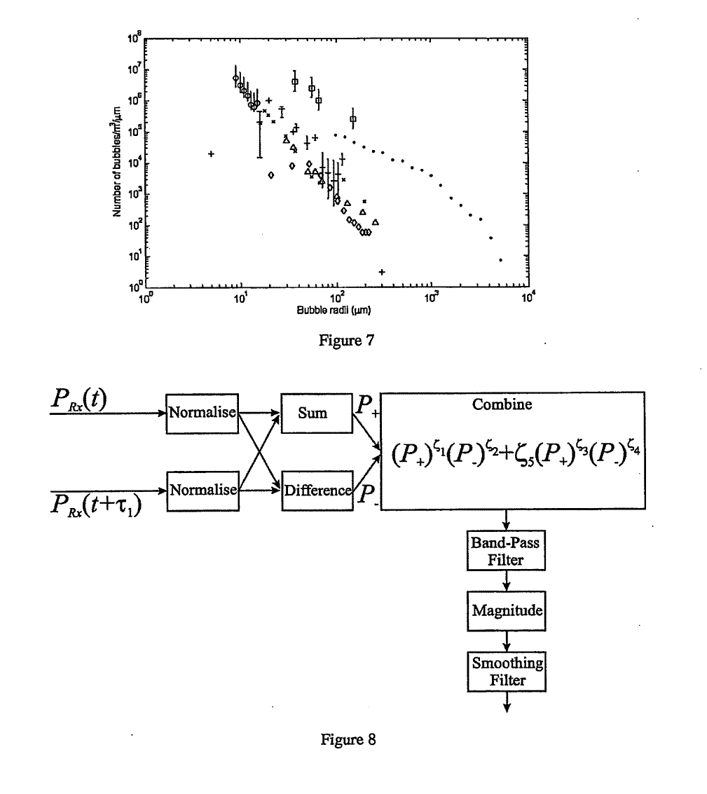

FIG. 7 Average bubble populations estimated by a number of key investigators. Surf zone data includes that collected by Meers et al. (2001; circles*), Leighton et al. (2004; bars*), Phelps et al. (1997; squares^{#}) and Deane & Stokes (1999; dots). Open ocean data includes that collected by Farmer et al. (1998; plus signs), Breitz & Medwin (1989; triangles), Johnson & Cooke (1979; diamonds) and Phelps & Leighton (1998; crosses).
* error bars indicate minimum and maximum recorded values
^{#} error bars indicate uncertainty due to the sampling volume

FIG. 8 is a schematic of some possible implementations for exploiting TWIPS1 and TWIPS2 signal processing. Note that this is not a unique solution, and that there are many options by which the basic ideas of TWIPS1 and TWIPS2 can be exploited. Whilst TWIPS1 is based on examination of, and comparison between, signals based on P₊ alone and P₋ alone, TWIPS2 is based on comparing mathematical combinations of P₊ and P₋ (of which, of course, there are an infinite number). In the combination procedure various different TWIPS operations are generated through the different choices of ζ₁, ζ₂, ζ₃, ζ₄ and ζ₅ (see Table 2). Other combinations (not shown in this figure for lack of space) include those where addition of a function or constant to the denominator of the ration is used to stabilise TWIPS2a. Such functions include those of the form 2P₋/(P₊+P₋) and P₋²/(P₊+P₋) (to improve the detection of the linear scatterers) and 2P₊/(P₊+P₋) and P₊²/(P₊+P₋) (to improve the detection of the nonlinear scatterers).

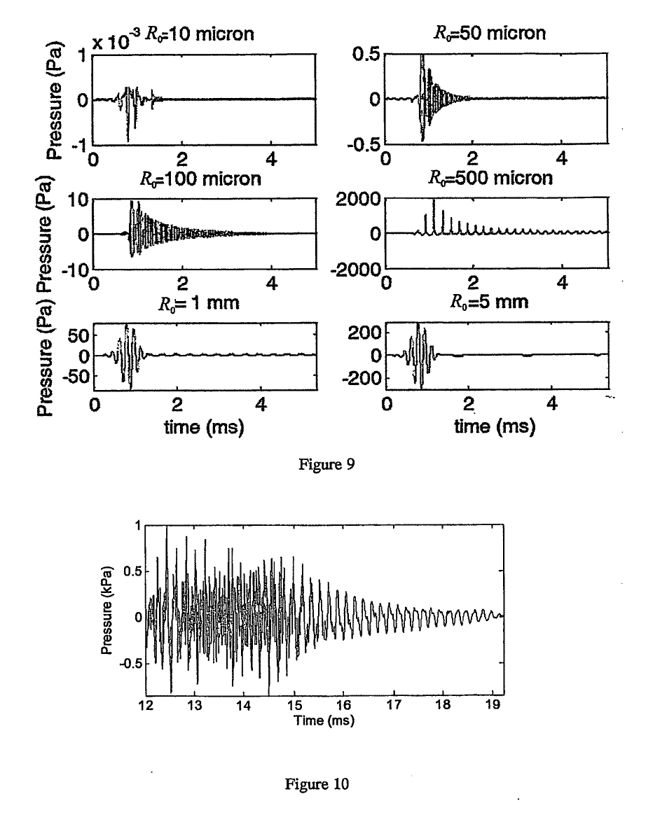

FIG. 9 The calculated pressures radiated at 1 m range from single bubbles of varying sizes in response to insonification by the ‘positive’ pulse only (the first pulse in FIG. 6(a)), which has a centre frequency of 6 kHz and a zero-to-peak acoustic pressure amplitude of 60 kPa. The equilibrium bubble radii R₀ chosen for the panels are 10 μm, 50 μm, 100 μm, 500 μm, 1000 μm, and 5000 μm.

FIG. 10 The simulated monostatic backscatter from the seawater containing a 1 m radius spherical bubble cloud containing, at its centre and 10 m from the transducer, a target which has target strength TS=−25 dB. The signals each show a typical return (‘positive’ pulse only) from a 6 kHz pulse. The signal from the target is buried is bubble noise, between 13.3 ms and 14.4 ms

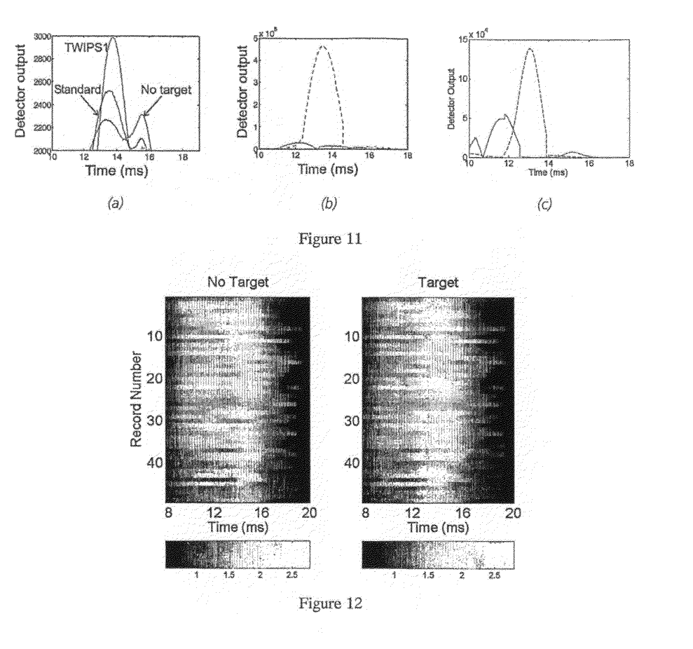

FIG. 11 Results of simulations of target detection by sonar, the data being taken from the scattering of a single pair of pulses. (a) Comparison of performances of TWIPS1 and “standard” sonar. TWIPS1 (ζ₁=0; ζ₂=1; ζ₃=ζ₄=ζ₅=0) has been applied at 6 kHz to a target of TS=−25 dB. The “standard result” was obtained by normalising the average return of two positive pulses from two different bubble clouds, and cross-correlating that output with the envelope of the input signal. The “No Target” plot was obtained by performing TWIPS1 on a cloud with no target. In (b), TWIPS2a (specifically, the version obtained in FIG. 8 when ζ₁=−1; ζ₂=1; ζ₃=ζ₄=ζ₅=0) has been applied to two cases: the bubble cloud on its own (solid line); the bubble cloud with a target of strength TS=−25 dB at its centre (dashed line). In (c), TWIPS2b (specifically, the version obtained in FIG. 8 when ζ₁=−1; ζ₂=2; ζ₃=ζ₄=ζ₅=0) has been applied to two cases: the bubble cloud on its own (solid line); the bubble cloud with a target of strength TS=−25 dB at its centre (dashed line). In all cases, the time scale has been shifted to account for the delay between the onset and maximum of the signal with which the time history has been convolved.

FIG. 12 Conventional sonar deconvolution techniques are deployed against the bubble cloud used in the simulation when it contains (on the left) no target, and (on the right) a target of TS=−20 dB. Fifty pulse pairs were projected at the cloud, spaced at intervals of 10 ms. Within the processing, a single average was formed from the two pulses that make up each pulse pair, such that 50 averages are available for plotting. Each average was plotted as a time history on a one-dimensional line, with a greyscale such that the amplitude of the signal at the corresponding moment in the time history was displayed: white corresponds to high detected amplitudes, and black corresponds to low detected amplitudes. These processed echo time histories were then stacked, one above each other, to form an image.

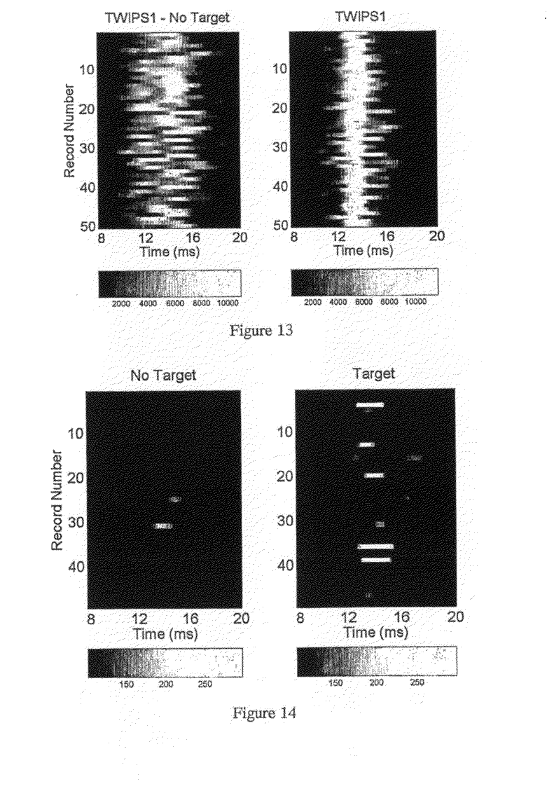

FIG. 13 TWIPS1 (ζ₁=0; ζ₂=1; ζ₃=ζ₄=ζ₅=0) is deployed against the bubble cloud used in the simulation when it contains (on the left) no target, and (on the right) a target of TS=−20 dB. Fifty pulse pairs were projected at the cloud, spaced at intervals of 10 ms. The TWIPS1 processed echoes were plotted, each as a time history on a one-dimensional line, with a greyscale such that the amplitude of the signal at the corresponding moment in the time history was displayed: white corresponds to high detected amplitudes, and black corresponds to low detected amplitudes. These processed echo time histories were then stacked, one above each other, to form an image.

FIG. 14 TWIPS2b (ζ₁=−1; ζ₂=2; ζ₃=ζ₄=ζ₅=0) is deployed against the bubble cloud used in the simulation when it contains (on the left) no target, and (on the right) a target of TS=−20 dB. Fifty pulse pairs were projected at the cloud, spaced at intervals of 10 ms. The TWIPS2b processed echoes were plotted, each as a time history on a one-dimensional line, with a greyscale such that the amplitude of the signal at the corresponding moment in the time history was displayed: white corresponds to high detected amplitudes, and black corresponds to low detected amplitudes. These processed echo time histories were then stacked, one above each other, to form an image.

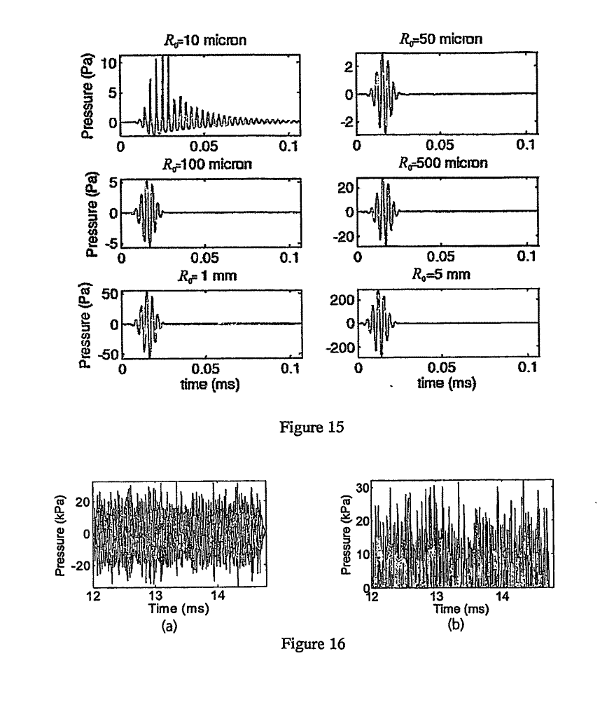

FIG. 15 The simulated responses from single bubbles equivalent to those in FIG. 9 for a 60 kPa pulse at 300 kHz (ie for insonification by the first pulse of the pair shown in FIG. 6(b)).

FIG. 16 Simulated scatter from a bubble cloud containing a target, the data being taken from the scattering of a single pulse (‘positive’ pulse only). (a) The time history returned from a bubble cloud containing a target TS=−25 dB at 10 m from the source processed using a 300 kHz tone burst of amplitude 60 kPa. (b) The root of the square of the signal from (a) is shown.

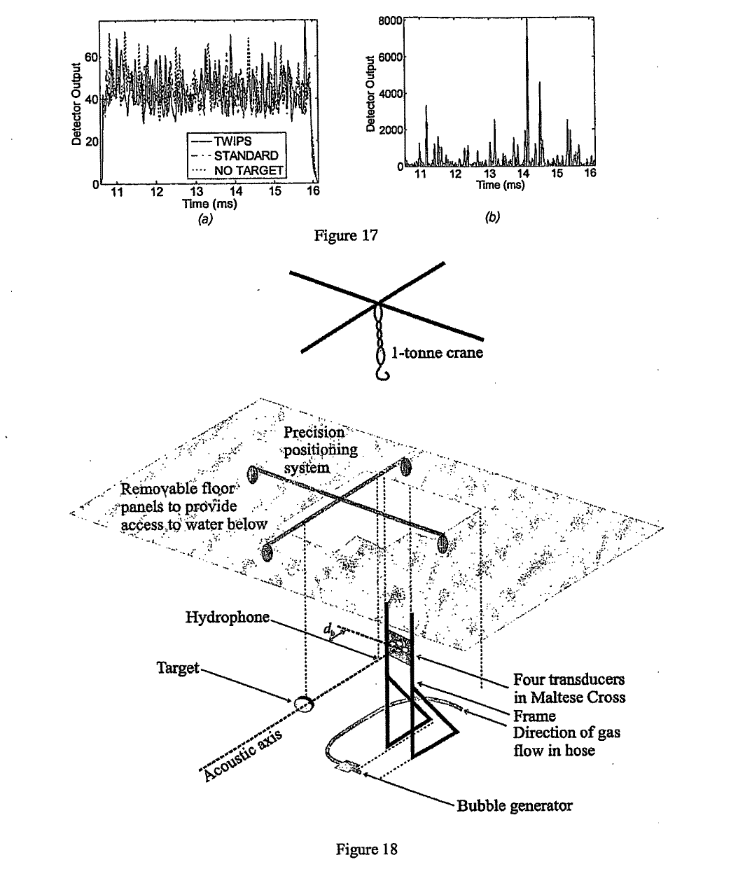

FIG. 17 Results of simulations of target detection by sonar, the data being taken from the scattering of a single pair of pulses. (a) TWIPS1 (ζ₁=0; ζ₂=1; ζ₃=ζ₄=ζ₅=0) has been applied at 300 kHz to a target of TS=−25 dB (solid line). It has been compared to the result obtained by ‘standard’ sonar, found by normalising the average return of two positive pulses from two different bubble clouds, and cross-correlating that output with the envelope of the input signal. The “No Target” plot was obtained by performing TWIPS1 on a cloud with no target. (b) TWIPS2a (ζ₁=−1; ζ₂=1; ζ₃=ζ₄=ζ₅=0) has been applied to the cloud containing the target of TS=−25. The target, if visible, would be found at 13.5 ms. As for the 6 kHz study above, the time scales have been shifted to account for the delay between the onset and maximum of the signal with which the time history has been convolved. Clearly it was impossible to detect the target using these techniques.

FIG. 18 Schematic of the apparatus for the experimental verification of TWIPS. The shaded plane corresponds to the floor of the laboratory, below which is an underground water tank measuring 8 m×8 m×5 m deep. Four transducers are mounted in a Maltese Cross, held on a rigid frame. Aligned on the acoustic axis are a hydrophone (at range dₕ=0.063 m from the source) and a removable target (at range 1.42 m from the source).

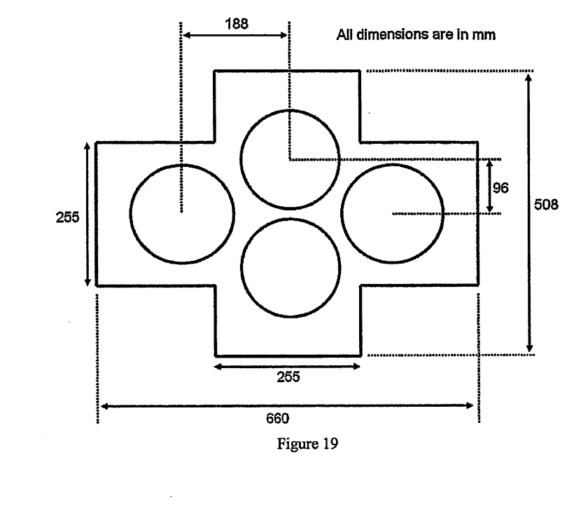

FIG. 19 Schematic of the dimensions of the Maltese Cross, as seen from along the acoustic axis. The circles correspond to the transducer faceplates, and the outer lines demarcate the edge of the rigid frame which holds the transducers.

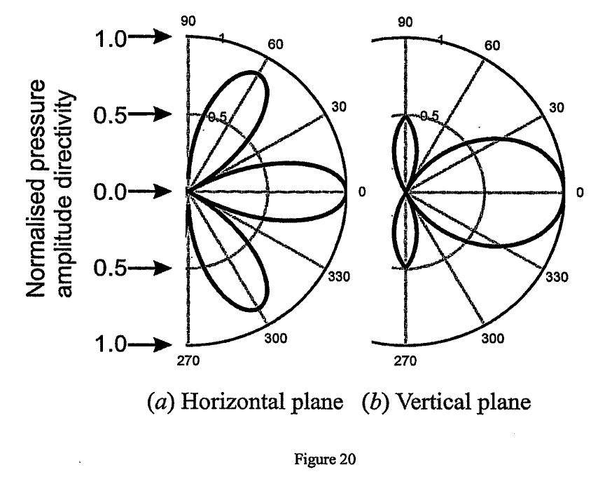

FIG. 20 Normalised amplitude far field directivity patterns of the 4-transducers in the Maltese Cross configuration at 6 kHz, for (a) the horizontal plane, and (b) the vertical plane, where the acoustic axis is at 0°. Plots provided courtesy of Ruth Plets.

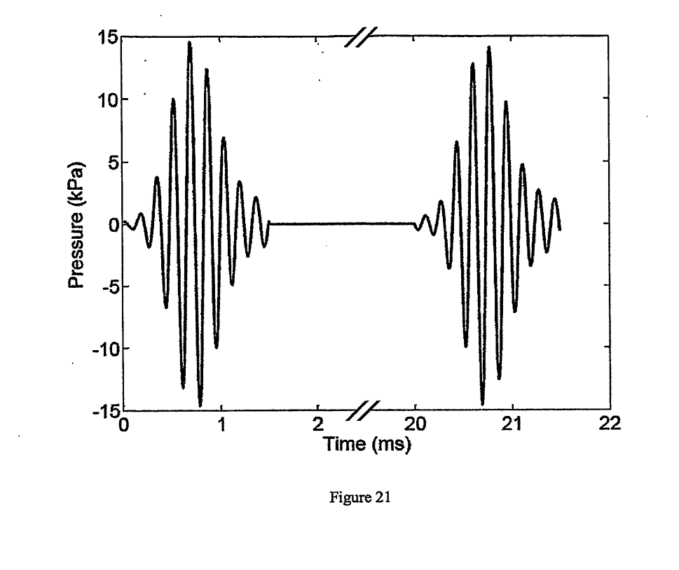

FIG. 21 The outgoing waveforms used for the TWIPS tests, measured on axis 63 mm in front of the transducer faceplate (see text).

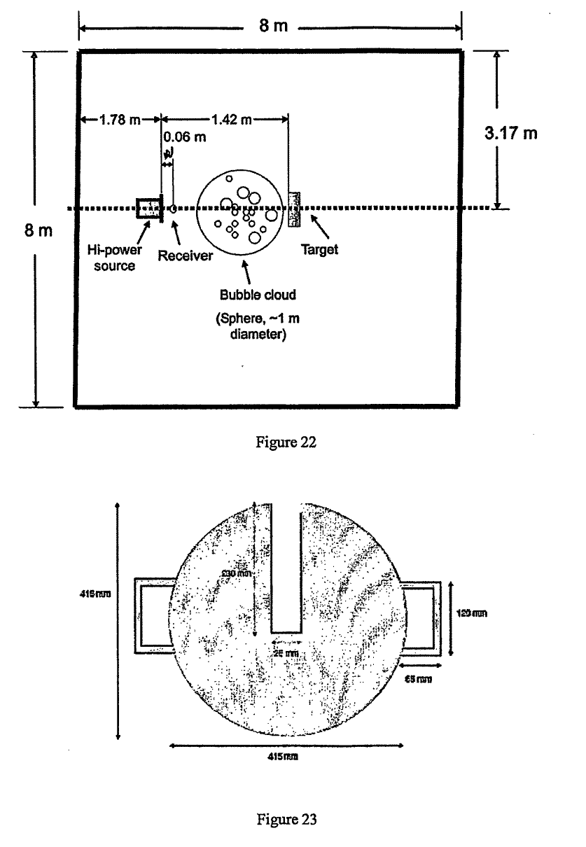

FIG. 22 Plan view of apparatus, showing length scales. The outer box indicates the perimeter of the water tank.

FIG. 23 Dimensions of the steel target. Its thickness out of the plane of the paper was 50 mm.

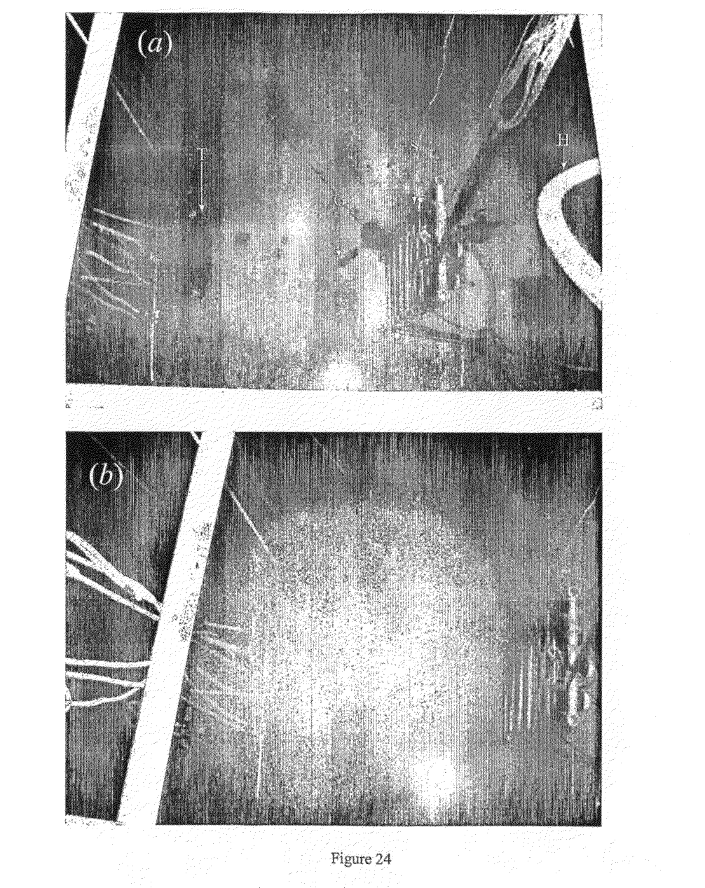

FIG. 24 (a) Photograph looking down into the water tank from above (the opposite direction to that shown in FIG. 22, so that the source is on the left), showing the target (T) and source (S). The hose (H) leads down to the bubble generator, whose tip is arrowed (G). The bubble cloud can just be seen forming in front of this tip. (b) A similar view to part (a), but here the target is obscured by the rising bubble cloud, which fills most of the space between the source and target, and in which the target is enveloped. The ropes upon which the target is suspended can be seen disappearing in to the cloud. The rig holding the source is still visible.

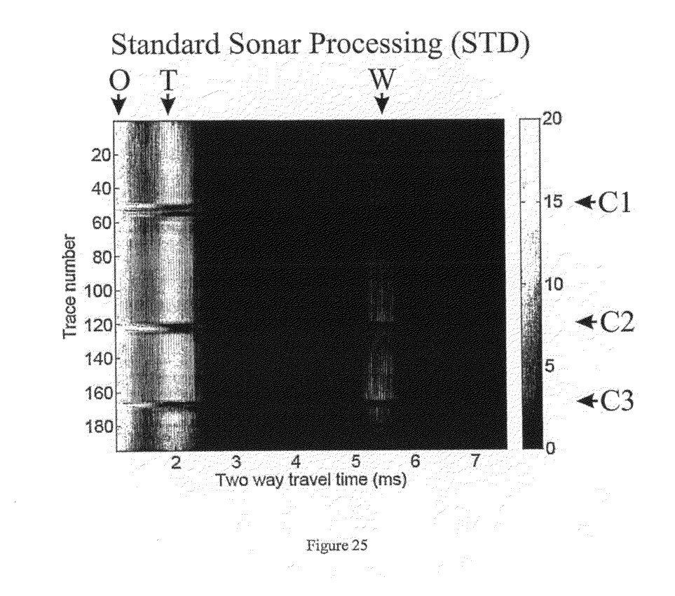

FIG. 25 The result of processing the TWIPS signal using standard sonar processing (described in text). A series of consecutive time histories recorded by the hydrophone are stacked, each labelled with a shot number (such that the earliest shots appear at the top of the figure). The range to the echoes is given in the two-way travel time (which does not of course apply to the outgoing pulse, which is centred on time zero and rings down shortly thereafter). The energy corresponding to the outgoing pulse (O) and the reflected signal from the target (T) are labelled. A weaker echo from the back wall is visible between 5 and 6 ms (labelled W). The passage of three bubble clouds through the sonar beam (labelled C1, C2 and C3) serves to hide the target and back wall.

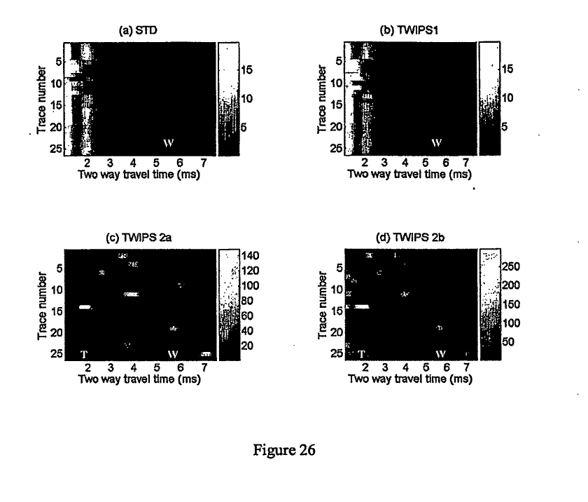

FIG. 26 A series of sonar echoes time histories is processed four ways, and then stacked. The four ways correspond to (a) Standard sonar processing (see text), (b) TWIPS1 (ζ₁=0; ζ₂=1; ζ₃=ζ₄=ζ₅=0), (c) TWIPS2a (ζ₁=−1; ζ₂=1; ζ₃=ζ₄=ζ₅=0), and (d) TWIPS2b (ζ₁=−1; ζ₂=2; ζ₃=ζ₄=ζ₅=0). The position in each time history of the target (T) and back wall (W) are shown. The bubble cloud passes the through the sonar beam during traces 4-15. The greyscale gives a linear representation of the detection algorithm output.

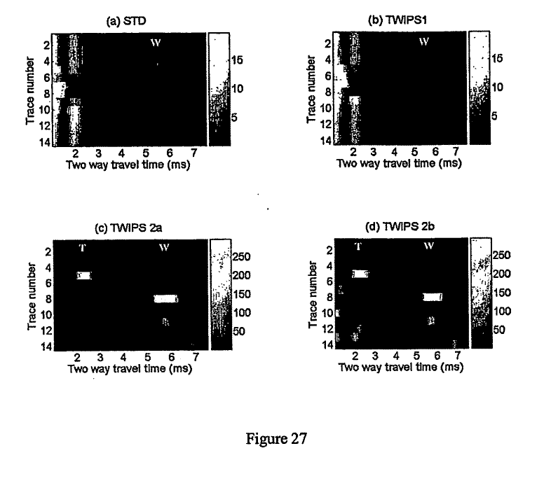

FIG. 27 A series of sonar echoes time histories is processed four ways, and then stacked. The four ways correspond to (a) Standard sonar processing (see text), (b) TWIPS1 (ζ₁=0; ζ₂=1; ζ₃=ζ₄=ζ₅=0), (c) TWIPS2a (ζ₁=−1; ζ₂=1; ζ₃=ζ₄=ζ₅=0), and (d) TWIPS2b (ζ₁=−1; ζ₂=2; ζ₃=ζ₄=ζ₅=0). The position in each time history of the target (T) and back wall (W) are shown. The bubble cloud passes the through the sonar beam during traces 5-9. The greyscale gives a linear representation of the detection algorithm output.

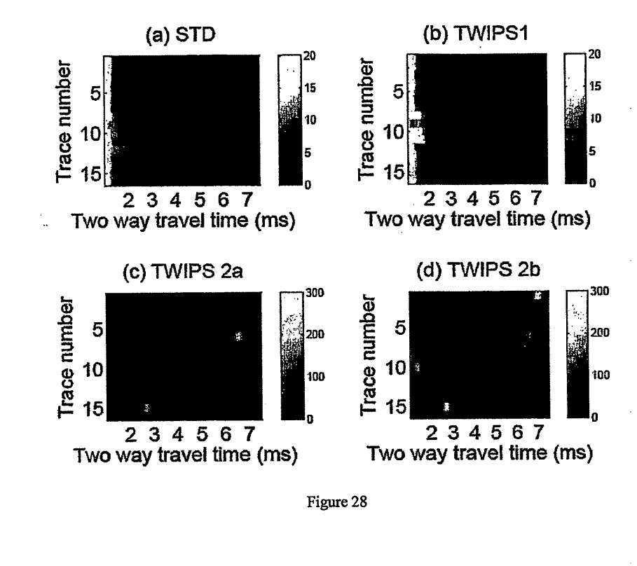

FIG. 28 A series of sonar echoes time histories, taken with no target present, is processed four ways, and then stacked. The four ways correspond to (a) Standard sonar processing (see text), (b) TWIPS1 (ζ₁=0; ζ₂=1; ζ₃=ζ₄=ζ₅=0), (c) TWIPS2a (ζ₁=−1; ζ₂=1; ζ₃=ζ₄=ζ₅=0), and (d) TWIPS2b (ζ₁=−1; ζ₂=2; ζ₃=ζ₄=ζ₅=0). The bubble cloud passes through the sonar beam during traces 7-12. The greyscale gives a linear representation of the detection algorithm output.

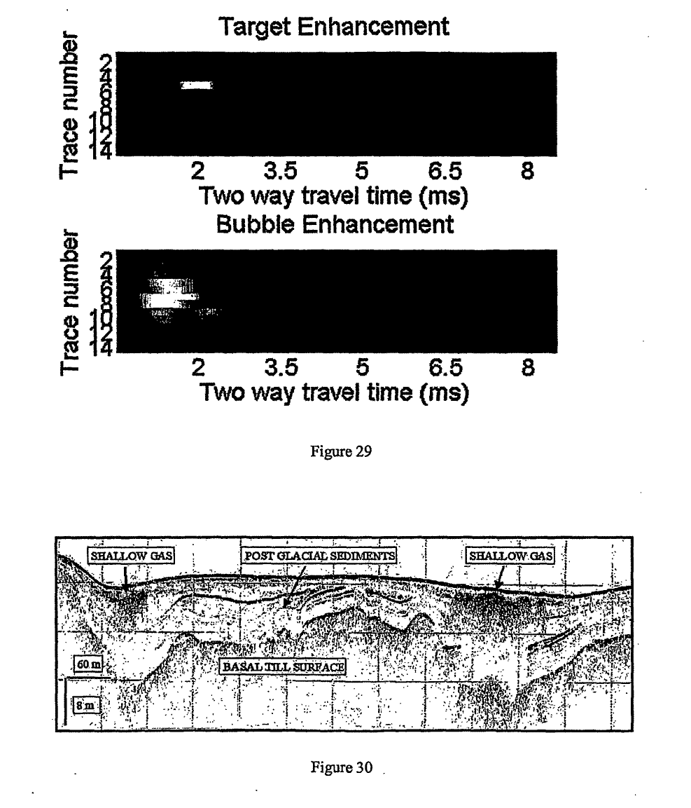

FIG. 29 A bubble cloud passes in front of the target (traces 4-11) and TWIPS2a processing is undertaken. In the upper plot TWIPS2a (ζ₁=−1; ζ₂=1; ζ₃=ζ₄=ζ₅=0) is used to enhance the target and suppress the bubble scatter. In the lower plot TWIPS2a (ζ₁=1; ζ₂=−1; ζ₃=ζ₄=ζ₅=0) is used to enhance the bubble scatter and suppress the scatter from linear objects.

FIG. 30 A traditional chirp sonar image, showing a cross-section of the seabed (maximum penetration approximately 20 m) in Strangford Lough, Northern Ireland. The dark line, which is usually 8-10 m from the top of the frame, indicates the sea floor. Hence the labelled features are beneath the seabed. These include shallow gas deposits in the underwater sediment. The sonar cannot penetrate these, as the majority of the sound is scattered from the gas bubbles. As a result, very little information is obtained from beneath the gas layers. Reproduced by permission of National Oceanography Centre, Southampton, UK (J. S. Lenham, J. K. Dix and J. Bull).

Acoustic systems (particularly sonar) have provided by far the most valuable sensors for use in an underwater environment. Shallow water and near-shore conditions can however considerably reduce their effectiveness. One environmental element which can compromise sonar is the presence of bubbles. These can be generated through biological and geophysical processes, but the overwhelming majority of bubbles are generated by wavebreaking. Near shore they can severely hinder the detection of targets, such as divers or mines (or fish, as shown in FIG. 1). We address this difficulty below by outlining how exploitation of the nonlinear behaviour of bubbles can ameliorate the operation of active sonar in bubbly environments (both in the water column and in gassy sediments). The technique has wider applicability, eg to target detection with other radiations.

There is currently a significant problem in the military community relating to the detection of mines in shallow coastal waters. In particular, bubbles created by breaking waves strongly scatter conventional sonar signals, masking scatter from the mines and making them very difficult to detect. This for example hampers the use of vehicles in shallow coastal waters.

The invention enhances the ability to detect such targets. Key to this is to ensure that enough of the energy scattered by the bubbles is scattered nonlinearly, whereas the energy scattered by the target (eg the mine) scatters linearly.

Nonlinear scattering, of course, may shift energy to higher frequencies.

Indeed because of this, even very rudimentary processing (such as band pass filtering) can enhance the contrast between the nonlinear bubbles and the linearly-scattering target once nonlinearities have been generated. (Note that, whilst insonification at sufficient amplitude close to resonance can excite nonlinearities in a near-monodisperse population, the presence of a wide range in bubble sizes (which can occur in the ocean) requires the use of low frequencies in addition to high driven amplitudes),

One example of contrast enhancement through rudimentary processing is as follows. If the receiver is narrowband, then energy scattered in harmonics above the fundamental by a bubble will, of course be ‘invisible’ to such a detector. If it is wideband, appropriate filtering can achieve the same effect, removing the energy scattered by the bubbles at higher harmonics from the detected signal. If the bubble population falls within a certain range of power law distributions, even a wideband receiver could detect sonar enhancements resulting from the reduced absorption which the bubble nonlinearity provides. Additionally, there may be further gains if more sophisticated processing is considered. These are described below.

FIGS. 2 and 3 illustrate one such route. The pulse of FIG. 2(a) is used to insonify a region of water containing both a linearly scattering target and an air bubble of radius 22.5 μm in water under 1 bar of static pressure. (Although FIGS. 2 and 3 simulate the insonification of a single bubble, recall the earlier discussion that a lower pulse frequency than the 65.7 kHz used in FIGS. 2 and 3 could be more effective at exciting nonlinearities from the oceanic bubble population if that population contains a wide range of bubble sizes, although this would be at the cost of the resolution afforded by the higher frequencies). All of the scattered waveforms in FIGS. 2(b), (c) and FIG. 3 are simulated at a distance of 1 m from the target and bubble. FIG. 2(b) shows the net scatter detected from the bubble and target. Whilst at first sight this may not seem to reveal much, when (in FIG. 2(c)) the scatter from the target alone (without the bubble, as calculated in FIG. 3(a)) is superimposed on the signal in FIG. 2(b), it is clear that the negative pressure component of the scattered signal more clearly shows the presence of the target than does the positive component. This is because the nonlinearity in the bubble response generates an asymmetry about the zero-pressure line, as will now be shown. Parts (b) and (c) of FIG. 3 show, respectively, the results when the signal in FIG. 3(a) (the scatter from the linear target alone) is subjected to positive and negative half wave rectification. The signal from the linearly scattering target contributes equally to both, such that the energy in FIG. 3(b) and (c) are equal. The nonlinearities in the scatter from the bubble create a different picture (FIG. 3(d)). The pressure scattered from the bubble alone (FIG. 3(d)) is clearly asymmetrical about the zero-pressure axis. Parts (e) and (f) of FIG. 3 show, respectively, the results when the signal in Figure 3(d) is subjected to positive and negative half wave rectification. The energy in FIG. 3(e) is more than 2.1 times greater than that in FIG. 3(f). This asymmetry of course provides a method by which the signal from the linearly scattering target can be distinguished from the bubble, if both contribute to the scattered signal (FIG. 2(b), (c)). Hence when (in FIG. 2(c)) the signal from the linearly scattering target (FIG. 3(a)) is superimposed on the signal of FIG. 2(b), the potential of the nonlinearity is clear: whilst the temporal peak in the positive pressure in the scattered signal of FIG. 2(b) comes from the bubble scatter, the temporal peak in the negative pressure comes from the linearly scattering target. Indeed FIG. 2(c) illustrates how much of the early stages of the return in FIG. 2(b) comes from the target. Of course, were the relative amplitudes of the scatter from target and bubble different, this simple result would not hold true, but the potential of the bubble nonlinearity to enhance the detection of targets and bubbles with respect to one another is clear.

Whilst illustrative, such examples should however be treated with care. There might, for example, be a temptation to quantify the enhancement in target detection by correlating the received signals with the driving pulse. However in FIGS. 2 and 3 the bubble is being driven close to half of its pulsation resonance frequency. The response from the bubble is almost entirely at the bubble resonance, whereas the response from the linear scatterer is at the frequency of the transmitted pulse. Hence it would be very easy to separate the linear from the nonlinear responses, simply by filtering about the bandwidth of the transmitted pulse (which causes the bubble response to vanish almost completely). This is of course exactly what a correlation process does. The correlation output, with or without rectification, would be dominated by the linear response. Hence a correlator would not help indicate any improvement obtained by rectification. Indeed one might argue that the detection analysis associated with the test in FIG. 3 should look at the response at the output of a correlator, since this is the minimum that a standard sonar system would employ. At the output of such a correlator you would not see an asymmetry in the waveform. This is because the correlator acts as a band-pass filter, with a fairly narrow pass band. To obtain asymmetry, the signal must have a spectrum that occupies more than an octave, which the output of a correlator will not, in general, achieve.

There is however a route to the exploitation of nonlinearities in enhancing target detection, which readily outperforms use of a standard correlator. This is here called Twin Inverted Pulse Sonar (TWIPS), which covers two basic subdivisions, TWIPS1 and TWIPS2 (of which there are a great number of forms). A schematic of how the preliminary stages of TWIPS operate is shown in FIG. 4, where the use of closely-spaced pulses of opposite polarity enhances the detection of a target (a fish, a mine etc.) with respect to bubbles. FIG. 4 illustrates just one of the ways in which the linear scatter from targets such as swim bladders driven off-resonance, or mines, might be enhanced compared to the scatter from oceanic bubble clouds. The pulse emitted by the transducer, P(t), has the following time series:

P(t)=Γ(t)−Γ(t−t₁) (1)

that is, a pulse containing two components based on a pressure function Γ(t); the second component starting a time t₁ after the first and having opposite polarity to it. An example of one such output is illustrated schematically at the top of FIG. 4 (below this, a vertical dashed line artificially divides FIG. 4 in half, such that the scattering from the bubble is shown separately on the left, and the scattering from the target is shown separately on the right). The signal returning to the receiver is a pressure wave denoted, P_{Rx}(t), which can be regarded as consisting of two components. The first component, Pₗ(t), is the result of linear scatters, for example mines and other rigid targets in sonar. The second component, Pₙₗ(t), arises from nonlinear scattering from objects, such as bubbles. Accordingly P_{Rx}(t) can be expressed as:

P_{Rx}(t)=Pₗ(t)+Pₙₗ(t) (2)

Assuming that it is the target that scatters signal linearly (FIG. 4(b)(i), then the contribution to the pressure detected by the sonar receiver that comes from the target is:

Pₗ(t)=s_{T}P(t−τ)=s_{T}(Γ(t−τ)−Γ(t−t₁−τ)) (3)

In this notation, and the following analyses, s_{T} is a constant scaling factor, and τ is the two-way travel time between the source/receiver and the scatterer. Linearly scattering structures may, of course, incorporate additional features, such as ring-up, ring-down and structural resonances. Whilst these will smear the target echo over time and so reduce the performance of a matched filter in both standard sonar and TWIPS, the innate linearity will nevertheless allow the initial stages of TWIPS (the formation of P₊ and P₋) to enhance contrast. The formulation could readily be adapted to include these additional features by representing s_{T} as an impulse response s_{T}(t) which is convolved with the pressure waveform P(t).

Suppose there is a bubble on which the same field is incident (FIG. 4(a)(i). The scattered signal is assumed to be nonlinearly related to the incident pulse such that the pressure contribution from it at the sonar receiver can be expressed as a power series (it is of course recognised that there are other descriptions of nonlinear responses, and that a power series expansion is not capable of describing all aspects of nonlinearity (eg subharmonics); it is however sufficient for the purpose of this illustration). Such a power series might take the following form, where for notational simplicity τ is assumed to be zero:

$$\begin{matrix}{P}_{nl} ( t ) = {s}_{1} P ( t ) + {s}_{2} {P}^{2} ( t ) + {s}_{3} {P}^{3} ( t ) + {s}_{4} {P}^{4} ( t ) + … \\ = {s}_{1} ( Γ ( t ) - Γ ( t - {t}_{1} ) ) + {{s}_{2} ( Γ ( t ) - Γ ( t - {t}_{1} ) )}^{2} + \\ {{s}_{3} ( Γ ( t ) - Γ ( t - {t}_{1} ) )}^{3} + {{s}_{4} ( Γ ( t ) - Γ ( t - {t}_{1} ) )}^{4} + …\end{matrix} \qquad ( 4 )$$

If the delay t₁ is sufficiently large so that Γ(t) and Γ(t−t₁) are never simultaneously non-zero (see below), then this equation simplifies to:

Pₙₗ(t)=s₁Γ(t)−s₁Γ(t−t₁)+s₂Γ²(t)+s₂Γ²(t−t₁)+s₃Γ³(t)−s₃Γ³(t−t₁)+s₄Γ⁴(t)+s₄Γ⁴(t−t₁)+L

[symbol]

Pₙₗ(t)=s₁Γ(t)+s₂Γ²(t)+s₃Γ³(t)+s₄Γ⁴(t)+ . . . −s₁Γ(t−t₁)+s₂Γ²(t−t₁)−s₃Γ³(t−t₁)+s₄Γ⁴(t−t₁)+ . . . (5)

TWIPS then combines this signal with a time-shifted version of itself. Considering the signal from a linearly scattering target, and subtracting time-shifted signals, one obtains:

P₋(t)=P_{Rx}(t)−P_{Rx}(t+t₁)=s_{T}(Γ(t)−(−Γ(t)))=2s_{T}Γ(t), 0≦t≦t₁ (6)

Note that the formation of the signal P₋(t)) can be implemented by convolving (filtering) the received signal by a filter with impulse response h(t)=δ(t)−δ(t+t₁). The amplitude of the signal P₋ (t) from the linear target is twice the amplitude of either of the original received components (FIG. 4(b)(iii)). Of course in practice the condition that Γ(t) and Γ(t−t₁) are never simultaneously non-zero could be violated. This would mean, for example, that the P₋ signal for a linear scatterer is not exactly twice the amplitude of either of the original received components. However such violations do not make TWIPS inoperable, but simply reduce the gain in these preliminary stages to less than a factor of 2.

When the same procedure is applied to the received signal from the nonlinearly scattering target, Pₙₗ(t), the amplitudes of the contributions from the linear and odd-powered nonlinearities are also enhanced (FIG. 4(a)(iii)). However the amplitudes associated with the even-powered nonlinearities of the scatter from the bubbles are suppressed (FIG. 4(a)(iii)). This means that the technique can be used to enhance the detection of linearly scattering targets compared to detection of bubbles. In general, it can be used to enhance the contrast of linear or odd-powered nonlinearities compared to even-powered nonlinearities. Of course, if the output is formed by adding the time-shifted signals, then the converse is true: the even-powered nonlinearities in the scatter from the bubbles are enhanced (FIG. 4(a)(ii)) and the radiation from the linear scatterer is suppressed (FIG. 4(b)(ii)), a technique used in pulse inversion biomedical ultrasonic contrast agent imaging.

In summary, by forming the signal P₋, as defined in (4), we can enhance the detection of linearly scattering targets with respect to bubbles. This initial stage of TWIPS is distinct from existing technology biomedical ultrasonic contrast agent imaging used for pulse inversion, which adds time-shifted versions of the signal to form P₊ in order to enhance the nonlinear scatter from bubbles. This can be expressed as:

P₊(t)=P_{Rx}(t)+P_{Rx}(t+t₁) (7)

However it is possible to take the technique further. If the ratio P₋/P₊ is formed, the detection of linear targets can be enhanced even further. Similarly if the ratio P₊/P₋ is formed, the detection of bubbles can be enhanced even further. There is of course a range of signals based on these possible combinations, such as P₊²/P₋² and P₋²/P₊². We shall call this use of the ratio TWIPS2. It enhances the contrast between the linear scatterers and the bubbles even further. Signals based on P₋/P₊, P₊/P₋, or powers of these ratios without stabilisation (see below) will be termed TWIPS2a. As an example, high values of P₋/P₊, which could potentially represent detection of the linear target(s), will constitute a series of large numbers, divided by series of small numbers. The bubble signals will not be enhanced to such a great extent. The opposite procedure (ie the formation of P₊/P₋) enhances the scattering of bubbles (eg contrast agents) with respect to, for example tissue: by dividing the addition signal by the subtraction signal, the scatter from the bubbles is greatly enhanced, which may have biomedical contrast agent applications. This could also be used for the detection of bubbles from diver breathing apparatus, or the ocean or seabed, or in pipelines (eg in manufacturing, harvesting or filling operations). Obviously the TWIPS2a technique needs to be applied carefully, because for example formation of the ratio can lead to a magnification of noise in the signal. The statistical distribution of noise on the output can exhibit highly non-Gaussian characteristics. In particular it will in general become more impulsive, which can lead to an increased false alarm rate. Were this to be the case, use of the ratio in TWIPS2a could be applied as a warning indicator, to alert the user to the possible presence of a target, which could then be examined for verification using the ordinary subtraction signal without taking the ratio. Alternatively, the TWIPS2a signal can be stabilised, forming one of the TWIPS2b or TWIPS2c functions, as will be discussed later. These warnings with respect to noise and false alarms having been stated, it should be noted that in the research results reported later, even the unstabilised TWIPS2a at times proved to be not particularly impaired by this feature.

Given now that there are ways of enhancing the contrast of bubbles with respect to targets, and vice versa, it is possible to make those contrasts stand out further by switching between subtraction and addition in TWIPS, or between P₋/P₊, and P₊/P₋ (or their equivalents) in TWIPS2. In this way, the ability to distinguish between linear and nonlinear scatterers would be further enhanced because of the ‘flashing’ effect between the two sets of images (FIG. 5).

There are an infinite number of ways of combining the P₊ and P₋ signals in TWIPS2. FIG. 8 shows some examples, although these can only be seen as representative of a wider range of combinations.

### Simulation of TWIPS

A simulation was developed in order to assess the potential for a TWIPS system to reveal a linearly scattering object in the presence of a bubble cloud. This section describes that simulation, the techniques used in processing the simulation output, and the results.

### Method

The simulation incorporates three primary inputs: a bubble cloud, a target, and an input acoustic signal. The signal returned by the bubble cloud is calculated, and then processed with the intention of revealing the presence of a linearly scattering object in the bubble cloud. The following assumptions were incorporated into the simulation: Bubble responses are uncoupled; The input sound pressure level is exactly the same at all points within the cloud; The cloud does not evolve during any single Twin Pulse; The time between Twin Pulses allows bubbles to move, but not dissolve; The target is assumed to displace no bubbles, has no acoustic shadow, and does not diffract any acoustic energy. Clearly several of these assumptions (such as the absence of pulse attenuation as it propagates through the cloud) can be refined at the expense of computational costs.

### The Target

It was assumed that the target would scatter linearly, in the manner described by equation (3). To find the level of the pulse returned by the target, a target strength was required. For the purposes of this simulation, the test target (which could in principle be a mine, a diver, etc) was chosen to be a fish. A target strength was selected, based on an acoustic model of the Atlantic cod (Gadus morhua). For initial studies of the effectiveness of TWIPS as a function of frequency, two characteristic carrier frequencies were selected: 6 kHz and 300 kHz, corresponding to the respective resonance frequencies for bubbles of radius 500 μm and 10 μm. In both cases, the cod was assumed to be broadside to the acoustic beam and assigned a target strength TS=−25 dB, equivalent to a fish of length 125 mm at 6 kHz and 330 mm at 300 kHz.

### The Bubble Cloud

The simulation developed for this study approximates a bubble cloud beneath a breaking wave. Meers et al. (2001) showed that the bubble population encountered beneath the breaking waves measured in their experiment can be approximated by:

n_{b}=6×10⁶e^{-0.02(R}^{₀}^{/1 μm}) (8)

where n_{b}(R₀)dR₀ is the number of bubbles per unit volume having a radius between R₀ and R₀+dR₀, and where R₀ (which must be expressed in microns for use in equation (8)) is the equilibrium radius of the bubble at the centre of each radius bin in a discretised bubble population.

To simplify the computing process, the entire bubble cloud was discretised and approximated as being comprised of bubbles within 5 logarithmically spaced radius bins with the following centre radii: 10 μm, 50 μm, 100 μm, 500 μm, 1000 μm, and 5000 μm. Using these centre radii and limits, equation (8) was found to give void fractions (the ratio of the volume of gas within a cloud to the total volume occupied by the cloud) on the order of 10⁻⁶ (ie 10⁻⁴%). The bubble population used to produce the simulation output presented in this paper is shown in Table 1:

TABLE 1
Bubble population used in the simulation.

| | | Number of bubbles in |
|---|---|---|
| Bubble radius | Size bin radius limits | size bin per cubic metre |
| (μm) | (μm) | of seawater |
| 10 | 10^{0.75} ≦ R₀ < 10^{1.25} | 3.5 × 10⁷ |
| 50 | 10^{1.25} ≦ R₀ < 10^{1.75} | 3.3 × 10⁶ |
| 100 | 10^{1.75} ≦ R₀ < 10^{2.25} | 3.0 × 10⁴ |
| 500 | 10^{2.25} ≦ R₀ < 10^{2.75} | 3.1 × 10² |
| 1000 | 10^{2.75} ≦ R₀ < 10^{3.25} | 3 × 10⁰ |
| 5000 | 10^{3.25} ≦ R₀ < 10^{3.75} | 0 |

The bubbles were randomly distributed within the perimeter of the cloud, but with no bubbles outside its spherical outer boundary. The object is to try to detect the target within the bubble cloud. In the model, this cloud does not evolve significantly in the 5 ms chosen for this simulation as the interval between a given ‘positive’ pulse and the subsequent ‘negative’ pulse. However after each ‘negative’ pulse, the cloud is allowed to evolve in keeping with known oceanic behaviour (with the restriction that the total number of bubbles in the cloud does not change). FIG. 1 shows the geometry of the problem.

### Ambient Noise

Oceans are very noisy environments. Surface waves, ship traffic, oceanic turbulence, seismic disturbances, marine mammals, and snapping shrimp are just a few of the many sources of sound that are distributed throughout the oceans of the world. The noises characteristic of such sources are varied both in temporal and spectral character, but can be approximated by the Wenz curves. The Wenz curves were devised to predict ambient noise in the ocean based on time-averaged representative noise spectra. However, given the high acoustic bandwidth of the system model (following from the high sampling rate), thermal noise is the dominant noise source. The effects of thermal noise Nₜₕₑᵣₘₐₗ were accounted for by using the relation:

N_{Thermal}=−15+20 log₁₀f (9)

where f is the bandwidth in Hz. The sampling frequency was used for f, and the noise was added to the simulated response before filtering and smoothing. Band-limited white noise was multiplied by the level N_{Thermal} to give the noise signal used for the simulation. Although temporally and spectrally inaccurate, this noise signal affects the signal processing in a manner to similar to real noise.

### The Radiated Pressures

The insonifying wavetrain is shown in FIG. 6. It consists of two pulses, each having an amplitude of 60 kPa (zero-to-peak), identical except that the second (the ‘negative’ pulse) has opposite polarity to the first (the ‘positive’ pulse). The frequency of the envelope and the carrier can be changed in the simulation. The operation of TWIPS is not dependent on this specific number of cycles in each pulse, nor on the envelope shape. Two specific simulations were performed, both with identical envelope shapes and number of cycles, but with different centre frequencies for the pulses: 6 kHz (FIG. 6(a)) and 300 kHz (FIG. 6(b)).

Generic bubble responses were found for bubbles of each radius using a modified nonlinear Herring-Keller equation. Once the displacement, velocity, and acceleration of the bubble wall are calculated, we seek the pressure radiated by the bubble. If the liquid can be treated as incompressible, then the pressure at any distance r from the bubble centre when the instantaneous bubble radius is R is:

$$( \frac{p}{{p}_{∞}} - 1 ) \frac{{p}_{∞}}{ρ} = \frac{R}{r} ( \ddot{R} R + 2 {\dot{R}}^{2} ) - \frac{{\dot{R}}^{2}}{2} {( \frac{R}{r} )}^{4} \qquad ( 10 )$$

where p_{∞} is the pressure in the liquid at some distance far enough from the bubble to be undisturbed by the excitation; and \dot{R} and \ddot{R} are respectively the velocity and acceleration of the bubble wall. The final term in equation (10) is related to the kinetic wave, which is normally treated as negligible at distances far from the bubble, although this should be critically examined when using such high amplitude pulses for target detection.

The relative amplitude of the echo from the linearly scattering target is given by a factor known as the Target Strength (TS). The degree to which the response by a bubble to a pressure perturbation is linear is primarily determined by the initial bubble size, the frequency of the input pulse with respect to that of the bubble resonance, and the amplitude of the input signal (plus factors of smaller importance such as surface tension, viscosity, etc.). The effectiveness of TWIPS increases in general as greater proportions of the bubble population scatter nonlinearly. If the population is monodisperse or near-monodisperse, then the greatest degree of nonlinearity (and hence the potential for TWIPS to work most effectively) tends to occur when the bubbles are driven at a frequency which is close to the main pulsation resonance of the population, or to some harmonic, subharmonic or ultraharmonic thereof. However many practical bubble populations contain a wide distribution of bubble sizes, and the solution is not so simple. If such populations are driven at a frequency which resonates with the bubble size that is found most commonly in the population, the degree of nonlinearity in the net echo from the population is diluted by the linear scattering from off-resonant bubbles (for example, large bubbles). For certain (but not all) populations, it is therefore necessary to select a different frequency to optimise the nonlinearity in the scattering from the population as a whole. Consider for example the oceanic bubble populations of FIG. 7. For oceanic populations, where a wide range of bubble sizes is present, such optimisation generally means that lower frequencies need to be used. This is because we need to give the bubble time to pulsate to large amplitude, and the characteristic response time of a bubble is determined by its own natural frequency. If the insonifying pulse is of high amplitude but high frequency (compared to the bubble pulsation resonance), then by the time the bubble has begun to respond to the first half cycle of the pulse (which, say, causes it to expand), it encounters the subsequent half cycle of the driving pulse (which in this example will tend to cause the bubble to contract). Therefore, the bubble simply does not respond rapidly enough to generate a highly nonlinear response if the driving sound field has a frequency much greater than its resonance. If however the bubble is sufficiently small that its natural frequency is much greater than the frequency of the driving pulse, it responds rapidly to the compressive or expansive half cycles, and undergoes high amplitude nonlinear pulsation.

This is a key consideration when faced with exploiting nonlinear bubble oscillations in the ocean, where there is a wide distribution of bubble sizes (ie bubbles having radii from microns to millimetres—FIG. 7). For a given pressure amplitude, by driving a bubble cloud at a low frequency, it is possible to excite more bubbles nonlinearly than by driving the same bubble cloud at a high frequency.

The resonance of a bubble can be approximated by:

$${ω}_{0} = \frac{1}{{R}_{0}} \sqrt{\frac{3 γ {p}_{i , e}}{{ρ}_{0}} - \frac{2 σ}{{ρ}_{0} {R}_{0}}} \qquad ( 11 )$$

where R₀ is the equilibrium radius of the bubble, γ is the polytropic index of the gas, p_{i,e} is the pressure within the bubble at equilibrium, ρ₀ is the density of the fluid surrounding the bubble, and σ is the surface tension of the liquid (more sophisticated versions include the effects of viscosity, vapour pressure etc.). According to equation (11), the resonance frequency of a bubble is approximately inversely proportional to the equilibrium bubble radius (true for air bubbles in water at Earth surface conditions for R₀>˜20 μm). Equation (8) indicates that, for a cloud of the type modelled here, the most populous bubbles are those that are smallest (on the order of tens of microns) (Table 1). Hence the majority of bubble resonances in a cloud are at high frequency (on the order of hundreds of kilohertz).

The obvious solution, when trying to exploit nonlinearities in bubbles, would be to argue that the bubble cloud should be driven at high frequencies (O(100 kHz)) in order to encourage nonlinear bubble pulsation. We will here show that this anticipated solution is however incorrect. Such an approach will drive the majority of the bubbles nonlinearly, but as explained, even at very high amplitudes of excitation (on the order of an atmosphere near the ocean surface) high frequency pulses will not drive large bubbles sufficiently nonlinearly. The huge number of bubbles in the system means that, if even only 1% of the bubbles in a spherical cloud of radius 1 m with a size distribution as described by equation (8) respond linearly, an algorithm searching for pulses of the type sent into the cloud will return several hundred thousand matches or more, and the desired target (mine, fish etc.) will be, masked.

This can be parameterised as follows. The operation of TWIPS depends on exciting nonlinearities in bubbles. It is of course understood that, for a given bubble and insonification frequency, an increase in the driving pressure will in general increase the wall pulsation amplitude and hence the degree of nonlinearity in the pulsation. Therefore one might reasonably expect that the operation of TWIPS requires high driving amplitudes (with the usual limitations with respect to generating cavitation at the transducer faceplate etc.). However the requirement to excite nonlinearity also has implications for the driving frequency. In short, for bubbles driven far from resonance, the lower the driving frequency, the greater the degree of nonlinearity in the bubble pulsation. Hence the performance of TWIPS with respect to generating nonlinearities in general will be better at O(kHz) than at O(100 kHz) for bubble populations of the type that might be found in the ocean a few metres below the sea surface.

The reason for this is that the lower frequency allows the bubble to grow to a larger size (normalised to the initial bubble radius). Why this is so can be understood in several ways. That low frequencies provide a longer rarefaction period in which to grow is only part of the answer. One must also consider the rates at which bubbles can respond to pressure field which would tend to make the bubble grow. The argument comparing the bubble pulsation resonance to the timescales for growth can now be formalised. The timescales over which large bubbles respond to pressure (i.e. grow during a rarefaction) are relatively slow compared to those of smaller bubbles (as evidenced by the approximately inverse relationship between bubble radius and natural frequency in equation (11)). This argument can even be extended to the regime of inertial cavitation (although of course such a high degree of nonlinearity is not necessary for the successful exploitation of TWIPS). Approximate analytical expressions for this time were given by Holland and Apfel (1989), who considered the delay times in bubble response for growth associated with inertial cavitation. They considered these delay times to be the summation of three components, corresponding to contributions caused by surface tension (Δt_{σ}), inertia (Δt₁) and viscosity (Δt_{η}), their sum being:

$$Δ {t}_{σ} + Δ {t}_{Ι} + Δ {t}_{η} \approx \frac{2 σ}{{P}_{A} - {P}_{B}} \sqrt{\frac{3 {ρ}_{0}}{2 ( {P}_{A} - {P}_{B} )}} + \frac{2 {R}_{0}}{3} \sqrt{\frac{{ρ}_{0}}{Δ {P}_{wall}}} + \frac{4 η}{Δ {P}_{wall}}$$

where P_{A} is the acoustic pressure amplitude of the insonifying field (assumed for the model of Holland and Apfel to be sinusoidal), and where P_{B} is the Blake threshold pressure, the degree of tension which must be generated in the liquid to overcome surface tension in allowing bubble growth:

$${P}_{B} \approx {p}_{0} + \frac{8 σ}{9} \sqrt{\frac{3 σ}{2 {R}_{0}^{3} ( {p}_{0} + 2 σ / {R}_{0} )}} , \qquad ( 124 )$$

and where ΔP_{wall} is the time-averaged pressure difference across the bubble wall:

ΔP_{wall}≈(P_{A}+P_{B}−2p₀+\sqrt{(P_{A}−P₀)(P_{A}−P_{B})})/3. (135)

If we consider the limitation associated with the growth of large bubbles, the issue is not with P_{B} (which becomes large for very small bubbles), but rather with other controlling timescales. In this large-bubble limit the dependence in equation Error! Reference source not found. of the time for growth on initial bubble radius is primarily through the inertial term Δt₁≈(2R₀/3)\sqrt{ρ₀/ΔP_{wall}}, which is approximately proportional to R₀. Therefore the larger the bubble, the more slowly it grows, and so during a given rarefaction cycle, the less the degree of growth it achieves. To put this another way, the maximum radius Rₘₐₓ achieved by a bubble during the growth phase of inertial cavitation is:

$${R}_{max} \approx \frac{4}{3 ω} ( P - {p}_{0} ) \sqrt{\frac{2}{{ρ}_{0} {P}_{A}}} {( 1 + \frac{2 ( {P}_{A} - {p}_{0} )}{3 {p}_{0}} )}^{\frac{1}{3}} \qquad ( 14 )$$

(Apfel, 1981; Leighton, 1994§4.3.1(b)(ii)) where ω is the circular frequency of the driving sound field. Equation (14) predicts that Rₘₐₓ will be independent of the initial bubble radius R₀. This point is in agreement with simulation and high speed photography—see FIGS. 4.8 and 4.19 of Leighton (1994). Whilst in FIG. 4.19 of Leighton (1994) several large bubbles (A,B,C,D) are seen pulsating throughout the figure, a host of bubbles which were initially too small to be seen (i.e. microscopic) grow in frame 4 to a size that is visible and of the same order as the large bubbles A, B, C and D. This is in agreement with FIG. 4.8 of Leighton (1994), where the ratio of the maximum size attained by the bubble during its oscillation to its initial size, increases for decreasing initial bubble size. If, as equation (14) predicts, the maximum size achieved by the bubble during the growth phase of inertial cavitation is independent of the initial bubble radius, then the scale of growth normalised to the initial radius (ie Rₘₐₓ/R₀) increases with decreasing bubble size. This further supports the idea that larger bubbles will require more time (as associated with a lower driving frequency) to achieve the same degree of nonlinearity in pulsation that would smaller bubbles. (Of course, whilst these arguments extend up to regimes where the bubble pulsation amplitude is sufficiently large for the phenomenon to be described as inertial cavitation, the successful operation of TWIPS by no means relies on such large pulsations, and indeed is also effective in the regime of non-inertial cavitation).

This explains why, for a population of small oceanic bubbles, a driving frequency of 1-20 kHz is more likely to excite the nonlinearity required of TWIPS than would 300 kHz at the same acoustic pressure amplitude. There are of course other factors which need to be included in consideration of the frequency chosen for sonar, including beam pattern, and spatial resolution. Other applications (biomedical ultrasonics, sonochemistry, electromagnetic systems including RADAR and LIDAR) will require commensurately different centre frequencies, pulse durations and separations. This is because of the different frequencies (and even radiations) and scatterers which are exploited in those applications: even in the use of biomedical ultrasonic contrast agents (which, like the example above, exploits acoustic radiation and a bubble-like population), the narrowness of the range of bubble sizes present means that sufficient nonlinearity can be generated by tuning the drive frequency closer to the resonance of most of the bubbles in the population, a technique which is far preferable to use of lower frequencies (with their commensurate loss of spatial and temporal resolution).

Therefore the degree of nonlinearity generated (key to the performance of TWIPS) in an oceanic bubble cloud is improved by use of a high amplitude low frequency driving pulse. The small bubbles will then be driven nonlinearly, as will the large bubbles. By then low-pass filtering the return from the cloud using a cut-off frequency just above the frequency of the input pulse, the extraneous high frequency information radiated from the nonlinearly excited bubbles will be diminished, and it becomes easier to search for a linear return from within the cloud. The details of such a search are given in the following section.

### Signal Processing

FIG. 8 shows a generic scheme for computing TWIPS outputs. The received signal, P_{Rx}(t), may be subjected to some signal conditioning, including normalisation. The two signals P₊ and P₋, which are the basis of the TWIPS processing, are formed by adding, and subtracting (respectively), the received signal with a delayed version of itself. The delay, t₁, matches the interval separating the outgoing pulses (although this is the best choice in most conditions, certain effects, such as inter-pulse perturbations in sound speed or Doppler effects, might make this choice suboptimal: compensation could be made, for example if the sonar source/receiver were travelling towards a stationary target at a known velocity).

The construction of P₊ and P₋ can be realised in a variety of fashions, including convolution with a signal consisting of a pair of Dirac delta functions, δ(t)±δ(t+t₁). The processing chain for TWIPS then combines the two signals P₊ and P₋ in a manner that emphasises either the linear or nonlinear components in the scattered signal, depending on the particular application. The various combinations are controlled by selection of the parameters ζ₁, ζ₂, ζ₃, ζ₄ and ζ₅ (FIG. 8). A band-pass filter is then applied. The final output of the systems is formed by constructing the envelope of the signal through smoothing the magnitude of the signal.

The pass bands of the two filters in the processing scheme are chosen in accordance with the properties of the combination stage. Wide band filters are generally more appropriate when the combinations used are nonlinear, whereas when using linear combinations of P₊ and P₋ one can employ filters with a narrow pass band.

The choices of ζ₁, ζ₂, ζ₃, ζ₄ and ζ₅ listed in Table 2 show some of the ways of implementing various TWIPS schemes, with example applications listed.

TABLE 2
Examples of how specific values for ζ₁, ζ₂, ζ₃, ζ₄ and ζ₅
can lead to ways to implement TWIPS schemes, with example
applications relating to sonar detection of mines, detection of
flotsam and fauna for collision avoidance, enhancement of detection
of biomedical contrast agents, reduction in ‘rusty bolt’ effect,
detection of covert or concealed circuitry for homeland security.

| ζ₁ | ζ₂ | ζ₃ | ζ₄ | ζ₅ | TWIPS | Example application |
|---|---|---|---|---|---|---|
| 0 | 1 | 0 | 0 | 0 | TWIPS1 | Some enhancement of linear targets |
| | | | | | | (e.g. fauna, mines) and suppression |
| | | | | | | of some nonlinear (e.g. bubble) |
| | | | | | | scatter |
| 0 | 2 | 0 | 0 | 0 | TWIPS1 | Some enhancement of linear targets |
| | | | | | | (e.g. fauna, mines) and suppression |
| | | | | | | of some nonlinear (e.g. bubble) |
| | | | | | | scatter |
| −1 | 1 | 0 | 0 | 0 | TWIPS2a | Enhancement of linear targets (e.g. |
| | | | | | | fauna, mines) and suppression of |
| | | | | | | some nonlinear (e.g. bubble) scatter |
| | | | | | | (potentially increased enhancement |
| | | | | | | compared to TWIPS1 but greater |
| | | | | | | instability) |
| −2 | 2 | 0 | 0 | 0 | TWIPS2a | Enhancement of linear targets (e.g. |
| | | | | | | fauna, mines) and suppression of |
| | | | | | | some nonlinear (e.g. bubble) scatter |
| | | | | | | (potentially increased enhancement |
| | | | | | | compared to TWIPS1 but greater |
| | | | | | | instability) |
| −3 | 3 | 0 | 0 | 0 | TWIPS2a | Enhancement of linear targets (e.g. |
| | | | | | | fauna, mines) and suppression of |
| | | | | | | some nonlinear (e.g. bubble) scatter |
| | | | | | | (potentially increased enhancement |
| | | | | | | compared to TWIPS1 but greater |
| | | | | | | instability) |
| 1 | −1 | 0 | 0 | 0 | TWIPS2a | Enhancement of nonlinear targets |
| | | | | | | (e.g. biomedical contrast agents with |
| | | | | | | ultrasound, or nonlinearly radiating |
| | | | | | | circuitry with EM) and suppression |
| | | | | | | of linear scatter (potentially |
| | | | | | | increased enhancement compared to |
| | | | | | | TWIPS1 but greater instability) |
| 2 | −2 | 0 | 0 | 0 | TWIPS2a | Enhancement of nonlinear targets |
| | | | | | | (e.g. biomedical contrast agents with |
| | | | | | | ultrasound, or nonlinearly radiating |
| | | | | | | circuitry with EM) and suppression |
| | | | | | | of linear scatter (potentially |
| | | | | | | increased enhancement compared to |
| | | | | | | TWIPS1 but greater instability) |
| −1 | 2 | 0 | 0 | 0 | TWIPS2b | Enhancement of linear targets (e.g. |
| | | | | | | fauna, mines) and suppression of |
| | | | | | | some nonlinear (e.g. bubble) scatter |
| | | | | | | (potentially greater stability than |
| | | | | | | TWIPS2a) |
| −1 | 3 | 0 | 0 | 0 | TWIPS2b | Enhancement of linear targets (e.g. |
| | | | | | | fauna, mines) and suppression of |
| | | | | | | some nonlinear (e.g. bubble) scatter |
| | | | | | | (potentially greater stability than |
| | | | | | | TWIPS2a) |
| 2 | −1 | 0 | 0 | 0 | TWIPS2b | Enhancement of nonlinear targets |
| | | | | | | (e.g. biomedical contrast agents with |
| | | | | | | ultrasound, or nonlinearly radiating |
| | | | | | | circuitry with EM) and suppression |
| | | | | | | of linear scatter (potentially |
| | | | | | | increased stability compared to |
| | | | | | | TWIPS2a) |
| 3 | −1 | 0 | 0 | 0 | TWIPS2b | Enhancement of nonlinear targets |
| | | | | | | (e.g. biomedical contrast agents with |
| | | | | | | ultrasound, or nonlinearly radiating |
| | | | | | | circuitry with EM) and suppression |
| | | | | | | of linear scatter (potentially |
| | | | | | | increased stability compared to |
| | | | | | | TWIPS2a) |
| −1 | 1 | 0 | 1 | 1 | TWIPS2c | Enhancement of linear targets (e.g. |
| | | | | | | fauna, mines) and suppression of |
| | | | | | | some nonlinear (e.g. bubble) scatter |
| | | | | | | (potentially greater stability than |
| | | | | | | TWIPS2a) |
| 1 | −1 | 1 | 0 | 1 | TWIPS2c | Enhancement of nonlinear targets |
| | | | | | | (e.g. biomedical contrast agents with |
| | | | | | | ultrasound, or nonlinearly radiating |
| | | | | | | circuitry with EM) and suppression |
| | | | | | | of linear scatter (potentially |
| | | | | | | increased stability compared to |
| | | | | | | TWIPS2a) |

### Results of Simulation

As the twin pulse signal is comprised of two pulses (‘positive’ and ‘negative’) in the simulation, it was necessary to calculate the bubble response for both portions. The response was then calculated from a region of seawater containing spherical cloud of bubbles of radius 1 m, centred on the target (which was at range 10 m from the transducer) (FIG. 1).

FIG. 9 shows the radiated pressures from the bin-centre bubble sizes in response to the positive portion of the 6 kHz twin wavetrain of FIG. 6(a). Bubbles of 500 μm radius or less clearly exhibit nonlinear behaviour. The larger bubbles (R₀>1 mm) respond almost linearly, and return a signal that is identical in form to the input pulse. In the computation, each pulse is comprised of 1600 points, giving a simulation resolution of 1.49×10⁶ samples/second. Note that choice of sampling frequency must take adequate account of the nonlinear distribution of energy to higher frequencies.

The simulation was then used to show the simulated monostatic backscatter from the seawater containing the bubble cloud, at the centre of which is the target. The signals analysed using TWIPS and shown in FIG. 11(a) and (b) were processed using the returns from a single pair of pulses. As TWIPS takes advantage of the returns from two pulses, a fair comparison with standard processing requires that the standard processor be allowed to average the return from two pulses before filtering and smoothing. As standard practice does not necessarily take advantage of the fact that bubble clouds evolve and thus degrade the ability for a standard correlation procedure to detect a target, the bubble cloud was allowed to evolve between pulses used for “standard” processing.

FIG. 10 illustrates the detection ability of acoustic backscattering, through examination of the scattered time history of the scattered pressure only. To do this, it shows the backscatter in response to the ‘positive’ pulse only (the average of 6 echoes is shown). The signal from the target is buried is bubble noise, between 13.3 ms and 13.5 ms. FIG. 11(a) demonstrates the use of standard sonar deconvolution techniques (which allow the target to be marginally detectable on this occasion) and the TWIPS procedure (which clearly identifies the target above the scatter from the cloud). The linear target, that was invisible in FIG. 10, is clearly identified by TWIPS1 as occurring between 13.3 ms and 13.5 ms.

Two options for TWIPS2 (TWIPS2a and TWIPS2b) were also tested (see the caption for the values of ζ₁, ζ₂, ζ₃, ζ₄ and ζ₅). These are defined through the processing shown in FIG. 8. In FIG. 11(b), the result of using TWIPS2a on the time history of FIG. 10 (that is, the same set of signals as were used to produce FIG. 11(a)) is spectacular in this case: the scatter from the target (which was invisible in FIG. 10) is now more than an order of magnitude greater than any scatter from the bubble cloud. It is however recognised, as discussed above, that this signal is less stable. FIG. 11(c) shows that the signal delivered by TWIPS2b processing also clearly shows the presence of the target above the scatter from the bubbles.

The implications for sonar imaging can be illustrated by plotting such time histories on a one-dimensional line, with a greyscale such that the amplitude of the signal at the corresponding moment in the time history was displayed: white corresponds to high detected amplitudes, and black corresponds to low detected amplitudes. For conventional sonar (FIG. 12), TWIPS1 (FIG. 13) and TWIPS2b (FIG. 14), 50 pulse pairs were projected at the cloud, spaced at intervals of 10 ms. The processed echoes were then stacked, one above each other, to form an image. As a stationary feature in the display, detection of the target in every ping would correspond to the observation of a vertical white line which is visible when the target is present, but absent from the corresponding sonar plot when the target is absent. The left hand plots in FIGS. 12 to 14 correspond to the cloud when there is no target present, and the right hand plots in FIGS. 12 to 14 correspond to the bubble cloud when the target (TS=−20 dB) is present. Standard sonar fails to detect the target: There is insufficient difference between the two plots which make up FIG. 12 because scatter from the bubbles masks the presence of the target. TWIPS1 detects the target on almost every occasion, such that there is a vertical line on the right of FIG. 13 compared to the plot on the left. As stated earlier, TWIPS2a works spectacularly when it detects a target, but it can be unstable. In FIG. 14, in that for some pings it fails to detect the target is present at all. However when it does detect one, the amplitude is very high (see plot on the right); when the target is not present (left hand plot), it rarely delivers a high amplitude return.

Of course, both TWIPS1 and TWIPS2 could be enhanced through exploitation with the Doppler signal generated when the scatterers are moving.

In contrast to the above, it can be seen that if the sonar utilises the normal frequencies exploited for oceanographic imaging (300 kHz is used in this example), then the linearly scattering target is undetectable amongst the bubble scatter. Indeed, the expected solution to generating high amplitude bubble pulsations in order to exploit bubble nonlinearities would be to use a high driving frequency of over 100 kHz. However the use of TWIPS with such frequencies is ineffective for the detection of linear targets obscured by bubble clouds in an oceanic environment having the wide range of bubble sizes used in this simulation.

This is demonstrated in FIG. 15. The same bubbles as were used for FIG. 9, are insonified with pulses identical to those used to generate FIG. 9, except that the centre frequency of each pulse is now not 6 kHz, but rather 300 kHz. FIG. 6(b) describes how the sets of pulses are identical in terms of the number of cycles and the peak acoustic amplitude. However it is clear that for all the bubbles in FIG. 14 excepting the smallest, the scatter is predominantly linear (that is, for all bubbles having radii in excess of 50 μm). This can be compared to the observation in FIG. 9 that only bubbles having radii greater than 1 mm produced primarily linear scatter.

As a consequence of this, when these higher frequencies are used, the sonar echoes are dominated by linear scattering from the oceanic bubble clouds. Because of this, TWIPS does not improve the ability to detect the target at all. Just as FIG. 10 demonstrated that in the raw time history of the echo, the target is not detectable when it is at the centre of the cloud and insonified at 6 kHz, so FIG. 16 shows that it is not detectable when insonified at 300 kHz.

Similarly, when the high frequency TWIPS1 technique is applied, it fails to detect the target hidden in the cloud (FIG. 17(a)). Indeed, without the additional factors described in this specification, even TWIPS2a fails to detect the target (FIG. 17(b)).

In fact, it will be seen that the methods developed in this current work are effective for small-target detection in this simulation only in the frequency range known in the ocean acoustics vernacular as ‘low frequency’. The reason for this is because of the “non-suppressed portion of the bubble signal”, which will now be discussed.

FIG. 4 makes clear that, when the two halves of the time series are subtracted, there is a considerable portion of the bubble signal which is not suppressed. This is, specifically, that relating to the linear and odd-powered nonlinearities, s₁(t)+s₃P³(t)+ . . . .

In order to make TWIPS work, the amount of the raw scattered signal which is invested in the linear needs to be reduced, and the amount in the even-powered nonlinearities need to be increased. The solution to this is counter-intuitive. It is to reduce the drive frequency from usual oceanographic imaging frequencies of 100 kHz or more, to what are considered to be low frequencies (say, a few kHz). If the frequency is too high, TWIPS will not work in an oceanic bubble population.

However, both TWIPS1 and TWIPS2 will work well at high frequencies in an environment, such as that prevalent in biomedical contrast agent imaging, in which all the bubbles are small and of a relatively uniform size. This is because very small bubbles do behave nonlinearly in response to a high frequency high amplitude pulse (see FIG. 15).

### Experimental Verification of TWIPS

Experiments were conducted to provide evidence of the performance of TWIPS, the scenario being the underwater detection of a mine-like target by sonar in a fresh water test tank. This tank, the A B Wood tank at the Institute of Sound and Vibration Research, University of Southampton, contains a body of fresh water measuring 8 m×8 m×5 m deep, with a water temperature of 11.2° C. and a sound speed (in bubble-free conditions) of 1449 m s⁻¹. The target was mounted along the acoustic axis of the sonar source, and bubble clouds could be generated at the base of the tank such that they rise in the buoyancy between the source and the target (FIG. 18).

### Method

The sonar source was rigidly mounted in the A B Wood tank, the source centre being at the depth of 2.8 m, with the acoustic axis horizontal (FIG. 18). The source consists of 4 individual transducers placed in a Maltese cross configuration (FIG. 19). In this configuration, the four transducers together made up a source having a main lobe full width half power beam width of approximately 30° in the horizontal plane, and 60° in the vertical plane (FIG. 20) at 6 kHz, the centre frequency of the TWIPS pulses. The acoustic axis was horizontal, and its depth (and that of the sources, receiver, and target) was 2.8 m. The on-axis zero-to-peak acoustic pressure amplitude of the signal (measured in the absence of the target and of any bubbles) was 38.08 kPa at a range of 1 m from the source faceplate, and 32.89 kPa at the position that would be occupied by the target. The acoustic data were taken at a hydrophone (Reson TC4013, Brookdeal Precision ac Amplifier type 9452) which was mounted on the acoustic axis of the source, and at a range of 0.063 m from it. The outgoing waveform measured by the hydrophone at that location is shown in FIG. 21, where the maximum zero-peak amplitude is 14.58 kPa. Although the results reported here use a TWIPS group which contains two pulses, preliminary tests were carried out with groups of three pulses (‘positive’, then ‘negative’, then ‘positive’). This was done for two reasons. First, it ensured that the two ‘positive’ pulses used for from the ‘standard sonar’ result were close together, to test whether using the two positive pulses from consecutive TWIPS pulses (as is done in the results presented below) unfairly downgraded the performance of the standard sonar as a result of bubble cloud evolution. It was found that it did not, so allowing two pulses to be used in the TWIPS group here. Second it was used for testing methods for optimising the delay t₁, although again such methods proved to be unnecessary in this test and t₁ was simply set equal to the interval between the two pulses.

Tests were conducted with and without a target in place, with and without a bubble cloud occupying space between the source and the target location. When present, the target was located at a range of 1.42 m from the source, centred on the acoustic axis (FIG. 22). The target, a steel mine mimic, is shown in FIGS. 23 and 24(a).

The bubble clouds had diameters of approximately 1 m to 2 m

(FIG. 24(b)), and contained bubbles ranging in radii from at least 10-1000 μm. At the depth of the target, the spatial average void fraction within the cloud was 7×10⁻⁶. It should be pointed out that the characteristics of the bubble cloud were only measured after the successful deployment of TWIPS reported here: this was not a case of using a priori information on the bubble cloud in order to optimise the insonification signal or the processing.

### Results of Experimental Tests

FIG. 25 shows the result of processing the TWIPS signal using standard sonar processing, which is implemented by band pass filtering P₊ and then computing a smoothed estimate of the envelope. A series of consecutive time histories recorded by the hydrophone are stacked, each labelled with a shot number. The energy corresponding to the outgoing pulse (O) and the reflected signal from the target (T) are labelled. The passage of three bubble clouds through the sonar beam (labelled C1, C2 and C3) serves to hide the target. The reflection from the back wall of the tank is faintly visible (W), after which come the returns from other multipaths.

FIGS. 26-28 show the signal from the hydrophone in four panes, generated by stacking time series as was done for FIGS. 12-14. Each figure shows a continuous sequence generated by consecutive TWIPS pulses, processed four ways (as indicated by the values of ζ₁, ζ₂, ζ₃, ζ₄ and ζ₅ in the caption) as the bubble cloud passes through the sonar beam. In FIGS. 26 and 27, the target is in place, whereas in FIG. 28 the target has been removed.

In FIG. 26(a), in the absence of the bubble cloud, standard sonar processing (STD) shows the outgoing pulse (at time zero), and the target (T), and a few weak reflections, the strongest of which comes from the back wall of the tank (W). However when the bubble cloud passes through the sonar beam (traces 4-15), the target and the wall are for the most part obscured. This is also true for TWIPS1 (FIG. 26(b)), although it is somewhat better at detecting the target, notably with an enhancement in ping 13. TWIPS2a shows echoes from the target (notably in trace 14), and also from the back wall, but there are false positives because of the instability in the algorithm (FIG. 26(c)). (Note that the two-way travel time to both will be slightly affected by the sound speed fluctuations caused by the bubble cloud). That it only detects the target once is not unexpected, in that the equipment available only allowed around a dozen pings at the target during the passage of the cloud. Similarly, the false positives resulting from the instability were expected. Both features have their basis in the inherent instability of the TWIPS2a method, as shown in the simulation, and require multiple pings properly to detect a target, and stabilisation to reduce the incidence of false positives. The latter effect is achieved in FIG. 26(d) where the strongest echo comes from the target. FIG. 26(d) also suggests that the ability to switch between and compare enhancement of the bubbles (or even standard sonar) with this TWIPS2b image (as suggested in FIG. 5) would highlight the presence of targets. Clearly the ability to increase the rate at which pings are generated would give more opportunities for TWIPS2 to detect the target during the passage of the cloud, and also improve the efficacy of the system (see later).

In FIG. 27, the cloud passes through the sonar beam in trace returns 5-9. Outside of this range, in bubble-free conditions, both Standard Sonar processing of the TWIPS pulses (STD, shown in FIG. 27(a)) and in TWIPS1 (FIG. 27(b)) clearly show the outgoing pulse (at time zero) and its ring-down as it passes the hydrophone. Both similarly show the echo from the target (T). The fainter signal at a two-way travel time of between 5 and 6 ms corresponds to the back wall of the tank (W). However when the bubble cloud passes through the sonar beam (returns 5-9), the target and back wall echoes are obscured in the plots of Standard Sonar (FIG. 27(a)) and TWIPS1 (FIG. 27(b)). The strongest return in both of these comes from the front of the bubble cloud between 0-1 ms, with very little signal detectable after this time window. However in FIGS. 27(c) and (d) respectively, both TWIPS2a and TWIPS2b clearly identify the target (T) and the back wall (W) (note again that the two-way travel time to both will be slightly affected by the sound speed fluctuations caused by the bubble cloud). The strongest echoes are from these objects, which also contribute weaker echoes in other traces. As expected from the simulation, not every ping is capable of detecting these linear scattering bodies: Ping 5 detects the target, and ping 8 detects the back wall. However when they fail to detect either, TWIPS2 gives false negatives, a trait which can be ameliorated by increasing the rate at which pings are generated (see below).

FIG. 28 shows the same four processing schemes and scenarios as did FIGS. 26 and 27 except that there is no target in place.

The above experiment has concentrated on detecting linear targets in the presence of bubbles. The detection of bubbles in the presence of linear targets is far easier, not only because of the strong scattering which results from bubble presence, but because of the summation features discussed in FIG. 4 (where addition of the pulses suppresses all of the linear components, in principle). However FIG. 29 demonstrates that TWIPS2 can also enhance bubble detection, which may be of use in finding buried gas pockets (i.e. buried fish with swim bladders or geophysical features).

The same dataset is processed by two different TWIPS2a schemes in the two panels of FIG. 29. The TWIPS2a scheme in the upper plot (ζ₁=−1; ζ₂=1; ζ₃=ζ₄=ζ₅=0) is designed, to enhance the linear scattering from the target but suppress that from the bubble cloud. Conversely, in the lower plot (ζ₁=1; ζ₂=−1; ζ₃=ζ₄=ζ₅=0) it is designed to enhance the bubble scatter and suppress that from the target. This could be used in the manner discussed in FIG. 5 to emphasise the presence of targets in bubble clouds, or to reveal the location of bubbles amidst linear scatterers. This could be applied to enhance the efficacy of ultrasonic contrast agent, or to detect gas reserves in sediment. FIG. 30 is a sonar image, generated through what is classed in this patent as standard (i.e. non-TWIPS) processing techniques. It reveals several ways in which TWIPS technology might be useful with respect to geophysical studies of gas in sediments. If TWIPS is used to enhance the scatter from bubbles and decrease the scatter from linear targets (e.g. sediment), then it could usefully be applied to detect gas pockets, such as those shown on the figure (as was done in FIG. 29). However the figure also shows that, such is the scatter from these gas pockets that no geophysical data can be obtained from beneath them: the area beneath the gas is white, indicating no signal can be obtained from below the gas; and beneath this white area, the darker patch is not in fact genuine geophysical data but rather a multipath artefact which shows up because of the strength of the scatter form the gas. The failure of the sonar to penetrate beyond the gas is of course very similar to the features in FIG. 25, and hence just as TWIPS allowed data to be obtained from beyond the cloud in FIGS. 26 and 27, so might TWIPS usefully be applied to geophysical problems such as that shows in FIG. 30.

### Conclusions

The experiment provided results very similar to those predicted by the simulation. Appropriate choice of the TWIPS algorithm could either enhance the detection of linearly-scattering targets in bubble clouds, or enhance the detection of bubble clouds. Oceanic applications include diver and mine detection, navigation etc. There are applications for contrast enhancement in biomedical or industrial situations. Having proved the technology, it potential for use with EM radiation (ameliorating the ‘rusty bolt’ effect, or detecting covert or hidden circuitry) is clear.

The chances of detecting the target using TWIPS are raised as the number of pulses emitted increases, provided that this increase does not complicate the detection of the target because of reverberation and clutter (which could, for example, make it difficult to identify which particular echoes to use in the detection algorithm). Best practice could ameliorate this difficulty by changing the characteristics (e.g. centre frequency) of the TWIPS pulses from one emission to the next.

### Concluding Remarks

The techniques in this specification describe an array of systems for enhancing the detection of linear scatterers with respect to nonlinear ones, and of enhancing nonlinear scatterers with respect to linear ones.

There are numerous applications. In the example above, the majority of the illustration scenarios were based on the sonar detection of linear scatterers (eg mines, fish) within bubble clouds, which can be made to scatter nonlinearly. This is a more difficult problem than that of enhancing the contrast of the nonlinear scatterers with respect to linear ones. Defense-related occurrences of the latter include the enhancement of the detection of the bubbles associated with diver breathing apparatus, propulsion systems and wakes, for example for harbour security. Example applications are discussed below.

Biomedical contrast agents: The use of pulse inversion at high frequencies has already been implemented to enhance the ultrasonic scatter from biomedical ultrasonic contrast agents with respect to tissue. This uses the process shown in FIG. 4, parts (a)(ii) and (b)(ii). However the scatter from contrast agents can be enhanced to a very much greater effect using the TWIPS2 method outlined in this report (the examples of TWIPS2a and TWIPS2b were given): rather than simply using the signal generated when the two halves of the time history are added, considerable extra enhancement can, for example, be obtained by then dividing this result by the signal obtained when the two halves of the time history are subtracted from one another. By enhancing the contrast of the agent, the amount of agent which needs to be injected into the body is reduced. This could have implications for both safety and cost.

Ultrasonic contrast agents have a range of applications. They usually consist of microscopic gas bubbles, injected into the body to enhance the scatter from blood. Since the agents move with the blood flow, they can also be used to assess such flow. Normally the acoustic impedance mismatch between blood and soft tissue is not great, and so the backscatter is not strong compared to the imaging of bone or gas bodies (for example in the gut). The ultrasonic imaging of blood flow can be greatly enhanced by ultrasonic contrast agents. Furthermore such agents have the potential to be used for therapy (for example, targeted drug delivery). Other examples of the acoustic detection of in vivo bubbles range from studies of decompression sickness to knuckle cracking and the detection of unwanted gas bubbles in blood vessel shunts.

As shown above, if the bubble population in question has a wide size distribution (as happens in the ocean and in many industrial environments—see below) then, for a given drive amplitude, reductions in the drive frequency are beneficial in increasing the nonlinearity in response of the population, because more of the large bubbles behave nonlinearly. In general, the wider the bubble population present, the more necessary it becomes to reduce the drive frequency. Again, this feature becomes especially important when trying to enhance the detection of linear scatterers which are being hidden by nonlinear scatterer (eg detecting mines hidden in bubble clouds), because TWIPS is far better at enhancing the nonlinear scatter with respect to the linear scatter than vice versa. Hence, the gains made by moving to lower frequencies when trying to enhance the scatter from biomedical contrast agents (which have a much narrower size distribution than is found in the ocean) are in most cases not enough to warrant the move, given that there would be a commensurate loss in spatial and temporal resolutions if lower frequencies were used. However in most cases of marine or industrial bubble problems, the move to lower frequencies would be more desirable (and in many cases vital) because of the large bubble size range present. This is particularly (but not exclusively) so when the problem is to enhance the detection of the linear scatterers which are hidden within nonlinear scatterers.

Industrial aspects of bubble detection: There are many scenarios in which the ability to enhance the scatter from bubbles, compared to linear scatterers, could be exploited. Industry contains many examples of the need for reliable bubble detection, management and control systems. In the petrochemical industry alone, for example, bubbles may be nucleated through the exsolution of gas which had, over time, dissolved into the crude oil in the high pressures at the well base, and which comes out of solution as the crude oil is brought up to surface pressures. Knowledge of the bubble population is required to optimise harvesting, transportation and safety. Bubbles entrained during filling operations involving molten glass or polymer solutions, or in the paint, food, detergent, cosmetics and pharmaceutical industries, may persist for long periods, degrading the product. Bioreactors, fermenters, and other biological processes in industry benefit from monitoring of the bubble population. Liquid targets for high energy physics, and coolant in power stations, would benefit from being monitored for bubble presence. In the pottery industry, liquid ‘casting slip’ is pumped from a settling tank, through overhead pipes and then into moulds for crockery, bathroom sinks, toilets etc. These are then fired in a kiln to make the finished product. If bubbles are present in the slip, these expand during firing, and ruin the product, a problem which is only discovered after firing has taken place. This means that the problem persists for many hours of production, wasting time, energy and materials (the fired pottery cannot be recycled). In all these examples, the ability to detect bubbles is hindered by the scatter from other objects (such as pipe walls, suspended solid particles etc). The use of nonlinearities as outlined in this report could dramatically increase the bubble detection abilities (for example through use one of the TWIPS2 variants). Conversely there are occasions when it would be preferable to use these techniques to reduce the scatter from the bubbles and enhance it from the linear scatters, such as when bubbles in the seabed hinder the penetration of sub-bottom sonar for geophysical or other examinations (FIG. 30).

Environment aspects of bubble detection: The ability to enhance the detection of bubbles is of importance to a number of environmentally significant processes as: the detection of those species of zooplankton which have associated gas bodies; coastal erosion and wave dynamics, methane seeps, and the fluxes between the ocean and atmosphere of momentum, energy and mass. The top 2.5 m of the ocean has a heat capacity equivalent to the entire atmosphere; and the flux between atmosphere and ocean of carbon alone exceeds 10⁹ tonnes/year.

LIDAR: Lidar (Light Detection And Ranging) has many uses, including atmospheric monitoring (where the wavelengths are appropriate to the sizes of aerosols, particles and other species which are to be investigated). There are several variants, including Doppler LIDAR and Differential Absorption Lidar (DIAL). Certain species, such as combustion products, can scatter LIDAR nonlinearly. Hence the application of the techniques of this report to LIDAR could enhance its ability to monitor for nonlinear scattering, with implications (for example) for environmental monitoring.

RADAR: RADAR can scatter nonlinearly from certain features (such as electrical circuitry). The so-called ‘rusty bolt’ effect arises in air gaps, of width 1-10 nm, in for example imperfect riveting or welding. Over time, the exposed metal surfaces are oxidised and metal-insulator-metal (MIM) junctions are formed. When these are exposed to RADAR or similar radiations, they can scatter nonlinearly as a result of electron tunneling through the insulator. The methods contained in this report could be used to detect such complex electrical phenomenon, whether their presence is intentional or not, by enhancing the scatter from the nonlinear components with respect to the linear ones. The applications could range from exploiting electromagnetic radiation of the correct frequency range to test weld strength or for crack detection, to allowing RADAR to detect complex electrical circuitry in possible targets. The presence of circuitry in such targets may be covert, with applications for homeland security. Alternatively, it might be used to suppress from the signal spurious ‘noise’ generated by such nonlinearities (in for example, radomes or antennae).

Other sensors: There are a range of sensors which produce nonlinear scatter, the enhancement of which (by the techniques outlined in this report) could be of importance. These include the nonlinear scatter of far infra-red radiation (eg for insect control and diseases diagnosis); laser scatter and spectroscopy, whereby elements in the sample may respond nonlinearly when exposed to high amplitude radiation; acoustic scatter for the detection of nonlinearly scattering inclusions in solids with applications to seismic sensors, borehole measurements, crack and fault detection, and the monitoring of corrosion, delamination or fatigue.

### REFERENCE LIST

Apfel R. E. Acoustic cavitation prediction. J. Acoust. Soc. Am., 69, 1624-1633 (1981).
Breitz N. and Medwin H., Instrumentation for in situ acoustical measurements of bubble spectra under breaking waves, J. Acoust. Soc. Am., 86, 739-743 (1989).
Deane G. B. and Stokes M. D., Air entrainment processes and bubble size distributions in the surf zone, J. Phys. Oc., 29, 1393-1403 (1999).
Farmer D. M., Vagle S. and Booth, A. D., A free-flooding acoustical resonator for measurement of bubble size distributions, J. Atmos. Oc. Tech., 15, 1132-1146 (1998).
Holland C. K. and Apfel R. E. An improved theory for the prediction of microcavitation thresholds. IEEE Transactions on Ultrasonics, Ferroelectrics, and Frequency Control, 36, 204-208 (1989).
Johnson B. D. and Cooke R. C. Bubble populations and spectra in coastal waters; a photographic approach, J. Geophys. Res., 84(C7), 3761-3766 (1979).
Leighton T. G., 1994. The Acoustic Bubble, London: Academic Press.
Leighton T. G., Meers S. D. and White P. R., Propagation through nonlinear time-dependent bubble clouds, and the estimation of bubble populations from measured acoustic characteristics. Proceedings of the Royal Society A, 460 (2049), 2004, 2521-2550.
Meers S. D., Leighton T. G., Clarke J. W. L., Heald G. J., Dumbrell H. A., and White P. R., The importance of bubble ring-up and pulse length in estimating the bubble distribution from propagation measurements, in Acoustical Oceanography, Proceedings of the Institute of Acoustics (editors: T G Leighton, G J Heald, H Griffiths and G Griffiths), 23(2), 2001, 235-241.
Phelps A. D., Ramble D. G. and Leighton T. G., The use of a combination frequency technique to measure the surf zone bubble population, J. Acoust. Soc. Am., 101, 1981-1989 (1997).
Phelps A. D. and Leighton T. G., Oceanic Bubble Population Measurements Using a Buoy-Deployed Combination Frequency Technique, IEEE J. Oc. Eng., 23(4), 400-410 (1998).

1. A method for creating an acoustic observation of a target volume, the method comprising the steps of transmitting a group of at least two acoustic pulses towards the target volume, receiving at least one detector an echo of the group scattered from the target volume, the echo having linear and nonlinear components, processing the scattered signal in such a way as to enhance at least part of the nonlinear component (and suppress the linear component) of the scattered signal in a signal P₊, processing the scattered signal in such a way as to suppress at least part of the nonlinear component (and enhance the linear component) of the scattered signal in a signal P₋, and producing a detection signal from a mathematical combination of the signals P₊ and P₋.

2. The method of claim 1 in which the mathematical combination comprises a ratio of said signals P₊ and P₋, or a function thereof, the combination being so chosen as to enhance or suppress at least part of the non-linear component, and suppress or enhance the linear component, according to requirements.

3. The method of claim 2 in which the mathematical combination comprises the ratio P₋/P₊ whereby some of the non-linear components are further suppressed and the linear components are further enhanced.

4. The method of claim 2 in which the mathematical combination comprises the ratio P₊/P₋ whereby some of the non-linear components are further enhanced and the linear components are further suppressed.

5. The method of claim 2 in which the mathematical combination comprises the product of the ratio P₊/P₋ with the numerator P₊ of the ratio.

6. The method of claim 2 in which the mathematical combination comprises the product of the ratio P₋/P₊ with the numerator P₋ of the ratio.

7. The method of claim 1 comprising switching between two different mathematical combinations in order to provide a contrast in the detection signal between two different levels of enhancement/suppression of the non-linear components of the scattered signal.

8. The method of any one of claims 1-7 in which the second pulse of the group of acoustic pulses is substantially identical to the first pulse but of opposite polarity, the acoustic signal being of the form P(t)=Γ(t)−Γ(t−t₁), where Γ is a pressure function, t is time, and t₁ corresponds to the time delay between the two pulses.

9. The method of claim 8 in which the time between the centre of a first pulse of the group and the centre of a second pulse of the group is greater than half of the characteristic decay time of the signal between the pulses.

10. The method according to any one of claims 1-7 applied to a target volume containing bubbles of a range of sizes, in which the driving frequency of the acoustic pulses is chosen to be lower than the resonance frequency of the majority of the bubbles.

11. The method of claim 10 applied to a target volume of water containing oceanic bubbles, the driving frequency of the acoustic pulses being less than 100 kHz.

12. The method of claim 11 in which the driving frequency of the acoustic pulses is less than 50 kHz.

13. The method of claim 12 in which the driving frequency of the acoustic pulses is less than 20 kHz.

14. The method of claim 11 in which the time delay t₁ between the pulses is greater than 10 μs.

15. Apparatus for creating an acoustic observation of a target volume, the apparatus comprising an acoustic pulse transmitter and an acoustic pulse receiver, a signal processing unit responsive to the output of the receiver, the signal processing unit being so configured as in use to enhance at least part of the nonlinear component (and suppress the linear component) of the scattered signal to produce a signal P₊, and also to suppress at least part of the nonlinear component (and enhance the linear component) of the scattered signal to produce a signal P₋, and a combiner unit arranged to produce in use a detection signal by mathematically combining the signals P₊ and P₋ in a manner such as to further enhance the contrast between said part of the nonlinear component and the linear component.

16. Apparatus for creating an acoustic observation of a target volume in a human or animal body, the apparatus comprising an acoustic pulse transmitter and an acoustic pulse receiver adapted to be positioned adjacent to a human or animal body, a signal processing unit responsive to the output of the receiver, the signal processing unit being so configured as in use to enhance at least part of the nonlinear component (and suppress the linear component) of the scattered signal from the target volume to produce a signal P₊, and also to suppress at least part of the nonlinear component (and enhance the linear component) of the scattered signal from the target volume to produce a signal P₋, and a combiner unit arranged to produce in use a detection signal by mathematically combining the signals P₊ and P₋ in a manner such as to further enhance the contrast between said part of the nonlinear component and the linear component.

17. A transmitting/receiving apparatus for observing a target by transmitting a pulsed electromagnetic signal towards the target and monitoring the receipt of signals scattered by the target, the transmitter being arranged to transmit a group of at least two pulses towards the target volume, the group of pulses being so configured that the scattered signal comprises linear and nonlinear components, the detector being arranged to process the scattered pulses resulting from said group in such a way as to modify the appearance of at least part of the nonlinear component of the scattered pulses in the receiver output signal.

18. Apparatus as claimed in claim 17 in which the electromagnetic signals are RADAR signals.

19. Apparatus as claimed in claim 17 in which the electromagnetic signals are LIDAR signals.

20. Apparatus as claimed in any one of claims 17-19 in which said part of the nonlinear component of the scattered electromagnetic pulses is suppressed (and the linear component enhanced) in the receiver output signal.

21. Apparatus as claimed in any one of claims 17-19 in which said part of the nonlinear component of the scattered electromagnetic pulses is enhanced (and the linear component suppressed) in the receiver output signal.

22. Apparatus as claimed in any one of claim 17, 18 or 19 in which a first receiver signal P₊ is produced by the receiver by processing the received scattered signal so as to enhance part of the nonlinear component (and suppress the linear component) of the scattered electro-magnetic pulses, and a second receiver signal P₋ is produced by processing the received scattered signal in such a way as to suppress at least part of the nonlinear component (and enhance the linear component), and a receiver output signal is produced from a mathematical combination of the signals P₊ and P₋ in a manner such as to further enhance the contrast between said part of the nonlinear component and the linear component.
